(19) United States
(12) Patent Application Publication
(10) Pub. No.: US 2026/0284128 A1
(43) Pub. Date: Sep. 24, 2026

(54) METHODS OF PRODUCING PENTACYCLIC TRITERPENOIDS FROM CULTURES VIA ELICITATION

(71) Applicant: Arkansas State University - Jonesboro, State University, AR (US)

(72) Inventors: Gaurav Gajurel, Jonesboro, AR (US); Luis Fabricio Medina-Bolivar, Memphis, TN (US)

(21) Appl. No.: 19/574,116

(22) Filed: Mar. 20, 2026

Related U.S. Application Data
(60) Provisional application No. 63/775,287, filed on Mar. 20, 2025.

(51) Int. Cl. A61K 36/185; A61K 31/56; A61P 31/04

(52) U.S. Cl. CPC A61K 36/185; A61K 31/56; A61P 31/04; A61K 2236/11; A61K 2236/35

(57) ABSTRACT

Provided herein are methods for producing pentacyclic triterpenoids comprising eliciting cultured hairy roots of Bixa orellana in a culture medium with an elicitor comprising methyl-β-cyclodextrin, methyl jasmonate, hydrogen peroxide, and magnesium chloride, and extracting pentacyclic triterpenoids from the elicited culture medium and, optionally, the elicited hairy roots. The pentacyclic triterpenoids include 2α,6β-dihydroxybetulinic acid, maslinic acid, oleanolic acid, 6β-hydroxybetulinic acid, arjunolic acid, betulinic acid, and alphitolic acid. Also provided are compositions comprising the triterpenoid-rich extract and methods of using the compositions for inhibiting induction of inflammatory markers, inhibiting growth or proliferation of bacteria, and inhibiting growth or proliferation of cancer cells.

### CROSS-REFERENCE TO RELATED APPLICATIONS

This application claims priority to U.S. Application No. 63/775,287, filed Mar. 20, 2025, which is hereby incorporated by reference in its entirety.

### BACKGROUND

Bixa orellana L., a member of the Bixaceae family, is native to South America and Central America. The B. orellana plant is commonly known as “achiote” in most Latin American countries and “annatto” in North America.^{1,2} It is widely cultivated for its medicinal properties and its use as a spice. B. orellana also holds economic value due to its applications in cosmetic, textile dye, and food industries.^{3,4} The plant's outer seed layer contains red to yellow pigments attributed to apocarotenoids, particularly bixin and norbixin.² The use of bixin for coloring cheese, ice cream, butter, edible oils, and bakery products has significant implications for the food industry.^{5,6}

Traditionally, the seeds of B. orellana have been used in treating heart-related diseases, diabetes, gonorrhea, and asthma.^{7,8} Additionally, its leaves are utilized for eye inflammation, bronchitis, sore throat, dysentery, and epilepsy, while the roots are used to treat fever and jaundice.^{8,9} B. orellana has also been reported to contain various specialized metabolites, including carotenoids, sterols, sesquiterpenes, flavonoids, and alkaloids.^{10,11} Terpenoids reported from B. orellana include arjunolic acid, stigmasterol, β-sitosterol, maslinic acid, ishwarane, and others.¹² These compounds exhibit a broad spectrum of bioactive properties, such as anti-inflammatory, antibacterial, antioxidant, antifungal, chemopreventive, antileishmanial, and anti-malarial effects.^{2,13-16}

### SUMMARY

The present disclosure relates to methods and compositions for producing pentacyclic triterpenoids from hairy root cultures of Bixa orellana through elicitation. The disclosed technology addresses challenges associated with obtaining bioactive triterpenoids in substantial quantities from conventional plant sources, which suffer from batch-to-batch variations and limited production of specialized metabolites. The hairy root culture system combined with the elicitation strategy disclosed herein provides genetic and chemical stability, enhanced triterpenoid yields, and efficient secretion of triterpenoids into the culture medium, thereby facilitating extraction and purification processes.

According to an aspect of the present disclosure, a method for producing pentacyclic triterpenoids is provided. The method comprises eliciting cultured hairy roots of Bixa orellana in a culture medium with an elicitor comprising methyl-β-cyclodextrin, methyl jasmonate, hydrogen peroxide, and magnesium chloride. The method further comprises extracting pentacyclic triterpenoids from the elicited culture medium and, optionally, the elicited hairy roots. The elicitor composition may comprise between 4.5-27 g/L methyl-β-cyclodextrin, between 1-150 μM methyl jasmonate, between 1.5-120 mM hydrogen peroxide, and between 0.5-10 mM magnesium chloride. The pentacyclic triterpenoids extracted from the elicited culture medium may include 2α,6β-dihydroxybetulinic acid, maslinic acid, oleanolic acid, and 6β-hydroxybetulinic acid. The extracted pentacyclic triterpenoids may further include arjunolic acid and, optionally, betulinic acid, alphitolic acid, or a combination thereof.

According to other aspects of the present disclosure, the method may include one or more of the following features. Eliciting the hairy roots may comprise eliciting for between 24 hours and 240 hours. The method may further comprise re-eliciting the hairy roots with the elicitor composition after extracting pentacyclic triterpenoids. The method may further comprise culturing the hairy roots in the culture medium for between 10 days and 20 days prior to elicitation. The method may further comprise purifying the extracted triterpenoids. The method may further comprise derivatizing one or more extracted pentacyclic triterpenoids. For example, one or more pentacyclic triterpenoids may be derivatized by acylation.

According to another aspect of the present disclosure, a composition is provided. The composition comprises elicited, cultured hairy roots of Bixa orellana, a culture medium, an elicitor, and pentacyclic triterpenoids. The elicitor comprises methyl-β-cyclodextrin, methyl jasmonate, hydrogen peroxide, and magnesium chloride. The pentacyclic triterpenoids comprise 2α,6β-dihydroxybetulinic acid, maslinic acid, oleanolic acid, and 6β-hydroxybetulinic acid. The pentacyclic triterpenoids may further comprise arjunolic acid and, optionally, betulinic acid, alphitolic acid, or a combination thereof. The composition may be prepared by eliciting cultured hairy roots of Bixa orellana in the culture medium with the elicitor according to the methods disclosed herein.

According to another aspect of the present disclosure, a triterpenoid-rich extract of the composition is provided. The extract comprises 2α,6β-dihydroxybetulinic acid, maslinic acid, oleanolic acid, 6β-hydroxybetulinic acid, and an extractant. The extract may further comprise arjunolic acid and, optionally, betulinic acid, alphitolic acid, or a combination thereof. The extractant may comprise ethyl acetate. The composition may further comprise a pharmaceutically acceptable carrier, excipient, or diluent.

According to another aspect of the present disclosure, a method of inhibiting induction of an inflammatory marker is provided. The method comprises contacting an immune cell with the triterpenoid-rich extract composition, a pentacyclic triterpenoid obtained from the composition, or a derivative of a pentacyclic triterpenoid obtained from the composition. The immune cell may be a macrophage. The inflammatory marker may be nitrite.

According to another aspect of the present disclosure, a method of inhibiting growth or proliferation of bacteria is provided. The method comprises contacting the bacteria with the triterpenoid-rich extract composition, a pentacyclic triterpenoid obtained from the composition, or a derivative of a pentacyclic triterpenoid obtained from the composition. The bacteria may be B. subtilis, E. coli, E. faecalis, E. faecium, K. pneumoniae, S. aureus, or S. mutans. The bacteria may be vancomycin-resistant E. faecium, vancomycin-resistant E. faecalis, or methicillin-resistant S. aureus.

According to another aspect of the present disclosure, a method of inhibiting growth or proliferation of cancer cells is provided. The method comprises contacting the cancer cells with the triterpenoid-rich extract composition, a pentacyclic triterpenoid obtained from the composition, or a derivative of a pentacyclic triterpenoid obtained from the composition. The cancer cells may be breast cancer cells.

### BRIEF DESCRIPTION OF FIGURES

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 depicts chemical structures of seven pentacyclic triterpenoids identified in elicited hairy root cultures of Bixa orellana.

FIG. 2 depicts a reaction schematic for semi-synthesis of acetylated derivatives of arjunolic acid and maslinic acid.

FIG. 3 depicts growth characteristics of Bixa orellana hairy root line 15 over a 30-day culture period. (A) Phenotype of hairy roots over the 30 days growth period. Each image is representative of triplicate cultures. (B) Change in the pH (dashed line) and conductivity (solid line) of the culture medium over time. (C) Growth curve of hairy roots. Each value represents the average of biological triplicates, and the error bars represent the standard deviation.

FIG. 4 depicts phenotypes of Bixa orellana hairy roots treated with different combinations of four elicitors for 192 h. MeJA, methyl jasmonate; CD, cyclodextrin; H₂O₂, hydrogen peroxide; MgCl₂, magnesium chloride.

FIG. 5 depicts HPLC chromatograms of culture medium from Bixa orellana hairy roots treated with different elicitor combinations. (A) Control (without elicitors); (B) CD+MeJA+H₂O₂+MgCl₂; (C) CD+MeJA+H₂O₂; (D) CD+MeJA; and (E) CD+H₂O₂. All the chromatograms were monitored at a wavelength of 200 nm. MeJA, methyl jasmonate; CD, cyclodextrin; H₂O₂, hydrogen peroxide; MgCl₂, magnesium chloride.

FIG. 6 depicts time-course dependent yields of four terpenoids in culture medium after elicitor treatment. (A) arjunolic acid, (B) 2α,6β-dihydroxybetulinic acid, (C) maslinic acid, and (D) oleanolic acid in the culture medium of Bixa hairy roots after treatment with different combinations of elicitors. Control (without elicitors) 1; CD+MeJA+H₂O₂+MgCl₂2; CD+MeJA+H₂O₂ 3; CD+MeJA 4; and CD+H₂O₂ 5. 2α,6β-dihydroxybetulinic acid was not detected in the control group 1 or hairy roots treated with CD+H₂O₂ 5. Maslinic acid and oleanolic acid were not detected in the control group. ‘n.d.’ represents 2α,6β-dihydroxybetulinic acid that was not detected in the hairy roots treated with CD+H₂O₂ for 192 h. The terpenoid yields are expressed in mg/L. Each bar represents the average of biological triplicates, and the error bars represent the standard deviation. Two-way ANOVA with Tukey's multiple comparison test was used for statistical analysis. MeJA, methyl jasmonate; CD, cyclodextrin; H₂O₂, hydrogen peroxide; MgCl₂, magnesium chloride.

FIG. 7 depicts total yield of terpenoids in culture medium and root tissue after elicitor treatment. The total yield of terpenoids in culture medium and root tissue after treatment of Bixa hairy roots with a combination of four elicitors (CD+MeJA+H₂O₂+MgCl₂) for 192 h. Each bar represents the average of biological triplicates, and the error bars represent the standard deviation. The yields were expressed as mg/g DW of roots.

FIG. 8 depicts yields of terpenoids in culture medium and root tissues of elicited and re-elicited hairy root cultures. The yields of arjunolic acid, 2α,6β-dihydroxybetulinic acid, maslinic acid, and oleanolic acid were measured in the culture medium and root tissues of B. orellana hairy root cultures treated with CD+MeJA+H₂O₂+MgCl₂ for 192 h, compared to those re-elicited for an additional 192 h. Each bar shows the average of three biological replicates, with the error bars representing the standard deviation. The yields were reported in mg per gram of root dry weight (DW). CD, methyl-β-cyclodextrin; MgCl₂, magnesium chloride; H₂O₂, hydrogen peroxide; MeJA, methyl jasmonate.

FIG. 9A depicts concentration-dependent cytotoxic effects of triterpenoids on RAW 264.7 cells. The concentration-dependent cytotoxic effect of triterpenoids on RAW 264.7 cells after 24 h treatment. The cytotoxic effects of triterpenoids were detected using the RealTime-Glo MT Cell Viability Assay reagent. Controls included cells treated with 0.05% DMSO. Each bar represents the average of biological triplicates, and the error bars represent the standard deviation. The dotted line represents the 50% cell viability. The arrow indicates the ordered arrangement of either (i) 2α,6β-dihydroxybetulinic acid, 6β-hydroxybetulinic acid, betulinic acid, and alphitolic acid; or (ii) arjunolic acid, maslinic acid, acetylated arjunolic acid, and acetylated maslinic acid.

FIG. 9B depicts concentration-dependent effects of triterpenes on growth inhibition of MCF-7 breast cancer cells. The concentration-dependent effect of triterpenes on growth inhibition of MCF-7 breast cancer cells. MCF-7 cells were treated with eight triterpenes at a concentration ranging from 3.12 to 100 μM for 72 h. The cytotoxic effects of triterpenoids were detected using RealTime-Glo MT Cell Viability Assay reagent. Cells treated with 0.05% DMSO were used as controls, wherein n=3. Each bar represents mean of three replicates+SD. The 50% cell viability is determined by the dotted line. The arrow indicates the ordered arrangement of either (i) 2α,6β-dihydroxybetulinic acid, betulinic acid, alphitolic acid, and 6β-hydroxybetulinic acid; or (ii) arjunolic acid, maslinic acid, acetylated arjunolic acid, and acetylated maslinic acid.

FIG. 10A depicts anti-inflammatory effects of triterpenoids in LPS-stimulated RAW 264.7 macrophages. The anti-inflammatory effects of triterpenoids were evaluated in lipopolysaccharide (LPS)-stimulated RAW 264.7 macrophages at concentrations of (A) 35 μM and (B) 20 μM for 24 h. Cells were pre-treated with terpenoids for 2 h before exposure to 1 μg/mL LPS for 24 h. Nitrite production was measured using Griess reagent and expressed as a percentage relative to the LPS-only treatment, which was set at 100%. Each bar represents the mean of three biological replicates, with error bars indicating standard deviation. Statistical analysis was conducted using Brown-Forsythe one-way ANOVA followed by Dunnett's post-test. Asterisks above the bars denote a significant decrease in nitric oxide levels compared to the LPS-only treatment (**p<0.002; ***p<0.001; ****p<0.0001).

FIG. 10B depicts effects of triterpenoid-rich extract on LPS-induced nitrite production in RAW 264.7 macrophages. Effect of triterpenoid-rich extract and the molar equivalent of triterpenes on LPS (lipopolysaccharide)-induced nitrite production in RAW 264.7 macrophages for 24 h. Cells were pretreated with triterpenoid-rich extract and the molar equivalent of triterpenes for 2 h before exposure to 1 μg/mL LPS for 24 h. Nitrite production was then measured using the Griess reagent system and expressed relative to LPS treatment alone, which was set at 100%. Each bar represents the average of three biological replicates, with error bars indicating standard deviation. Statistical analysis was performed using Brown-Forsythe one-way analysis of variance (ANOVA) with Dunnett's post-test. Asterisks above the bars indicate a significant reduction in nitric oxide levels compared to LPS treatment alone (*, p<0.05; **, p<0.002; ***, p<0.001; *: **, p<0.0001).

FIG. 11 depicts a semi-preparative HPLC chromatogram for purification of triterpenoids from silica gel column chromatography fractions.

FIG. 12 depicts a semi-preparative HPLC chromatogram for purification of triterpenoids from ODS column chromatography fractions.

### DETAILED DESCRIPTION

Provided herein are methods and compositions for producing pentacyclic triterpenoids from hairy root cultures of Bixa orellana through elicitation. The methods comprise eliciting cultured hairy roots with an elicitor composition comprising methyl-β-cyclodextrin, methyl jasmonate, hydrogen peroxide, and magnesium chloride, and extracting pentacyclic triterpenoids from the elicited culture medium and, optionally, the elicited hairy roots. The pentacyclic triterpenoids produced include 2α,6β-dihydroxybetulinic acid, 6β-hydroxybetulinic acid, arjunolic acid, maslinic acid, oleanolic acid, betulinic acid, and alphitolic acid. The methods and compositions disclosed herein provide several advantages over conventional approaches for obtaining bioactive triterpenoids. The hairy root culture system offers genetic and chemical stability, enabling consistent production of specialized metabolites without the batch-to-batch variations associated with field-grown plants. The elicitation strategy enhances triterpenoid yields, e.g., achieving up to 71.5 mg/g root dry weight, with re-elicitation providing an additional 36% increase in yield. The majority of the triterpenoids are secreted into the culture medium, facilitating extraction and purification. The triterpenoids and triterpenoid-rich extracts obtained by the disclosed methods exhibit valuable bioactivities, including anti-inflammatory effects, antibacterial activity against drug-resistant pathogens such as vancomycin-resistant Enterococcus and methicillin-resistant Staphylococcus aureus, and cytotoxic effects against cancer cells. Semi-synthetic acetylated derivatives of arjunolic acid and maslinic acid demonstrate enhanced anti-inflammatory activity compared to their parent compounds.

Bixa orellana L., a South American plant valued for its medicinal and culinary uses, was studied using hairy root cultures to produce bioactive pentacyclic triterpenoids. Hairy root cultures were elicited with an elicitor composition comprising methyl-β-cyclodextrin, hydrogen peroxide, magnesium chloride, and methyl jasmonate. The Examples demonstrate the presence of 2α,6β-dihydroxybetulinic acid and 6β-hydroxybetulinic acid in B. orellana along with five known pentacyclic triterpenoids: arjunolic acid, maslinic acid, oleanolic acid, betulinic acid, and alphitolic acid. Elicitation increased the yield of these triterpenoids with a further increase upon re-elicitation. Semi-synthetic acetylated derivatives of arjunolic and maslinic acids were also synthesized. Maslinic acid exhibited antibacterial activity against vancomycin-resistant and methicillin-resistant bacteria. This demonstrates the potential of B. orellana hairy roots as a sustainable platform for producing bioactive pentacyclic triterpenoids.

Although B. orellana is a candidate for producing bioactive specialized metabolites,¹⁷ obtaining these compounds in substantial quantities is challenging due to variations between batches and the limited production of specialized metabolites.^{18,19} In recent years, hairy root cultures obtained via Agrobacterium rhizogenes-mediated transformation have emerged as a sustainable platform to produce these specialized metabolites. This biotechnological platform exhibits genetic and chemical stability, rapid growth, and the ability to produce metabolites in large amounts.²⁰

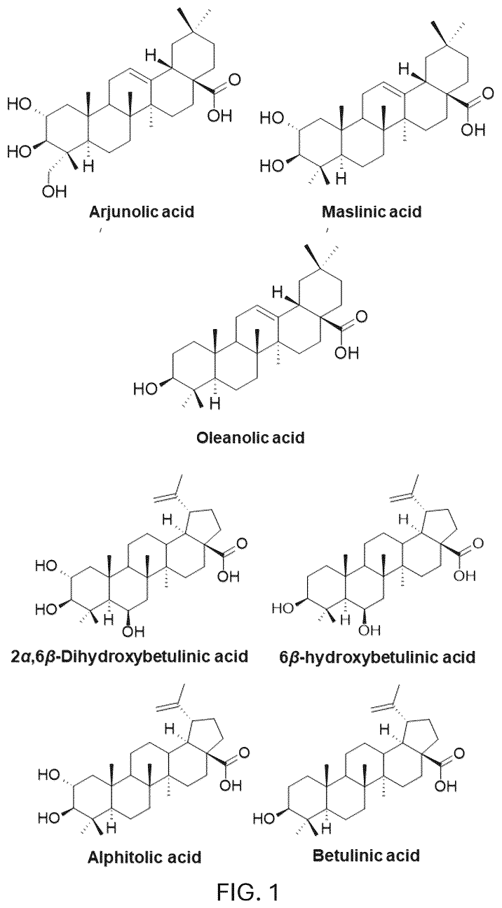

Seven pentacyclic triterpenoids were identified in the elicited culture medium of B. orellana hairy roots elicited with an elicitor composition comprising MeJA, methyl-β-cyclodextrin (CD), hydrogen peroxide (H₂O₂), and magnesium chloride (MgCl₂). These compounds were isolated by column chromatography and semi-preparative HPLC (FIG. 1). Among these, 2α,6β-dihydroxybetulinic acid and 6β-hydroxybetulinic acid were reported for the first time in the genus Bixa, and their structures were confirmed using mass spectrometry (MS) and nuclear magnetic resonance (NMR) spectroscopy. The Examples achieved high yields of arjunolic acid, 2α,6β-dihydroxybetulinic acid, and maslinic acid, which were enhanced by employing a re-elicitation strategy for sustainable production. Additionally, acetylated derivatives of arjunolic acid and maslinic acid were synthesized via acetylation reactions. The anti-inflammatory, cytotoxic, and antibacterial activities of the natural and semi-synthetic compounds were also evaluated.

Referring to FIG. 1, seven pentacyclic triterpenoids were identified in the medium of elicited hairy root cultures of Bixa orellana. These pentacyclic triterpenoids may be classified into two structural types: oleanane-type triterpenoids and lupane-type triterpenoids. Oleanane-type triterpenoids identified include arjunolic acid, maslinic acid, and oleanolic acid. Lupane-type triterpenoids identified include 2α,6β-dihydroxybetulinic acid, 6β-hydroxybetulinic acid, alphitolic acid, and betulinic acid.

The oleanane-type and lupane-type triterpenoids differ in the arrangement of the E-ring, with oleanane-type compounds featuring a six-membered E-ring and lupane-type compounds featuring a five-membered E-ring with an isopropenyl substituent. The number and position of hydroxyl groups on the triterpenoid backbone varies among the compounds, which may influence the bioactivity profiles of the individual triterpenoids.

The methods disclosed herein comprise eliciting cultured hairy roots of Bixa orellana in a culture medium with an elicitor comprising methyl-β-cyclodextrin, methyl jasmonate, hydrogen peroxide, and magnesium chloride. The elicitation process induces the production and secretion of pentacyclic triterpenoids into the culture medium.

The elicitor composition may comprise methyl-β-cyclodextrin (CD). Methyl-β-cyclodextrin may serve a dual role in the elicitor composition. As an elicitor or inducer, methyl-β-cyclodextrin may activate the production of pentacyclic triterpenoids in the hairy root cultures. As a trapping agent, methyl-β-cyclodextrin may prevent feedback inhibition, which may lead to the accumulation of pentacyclic triterpenoids at high levels. In some cases, the methyl-β-cyclodextrin concentration ranges from about 4.5 g/L to about 27 g/L, about 10 g/L to about 25 g/L, or about 15 g/L to about 20 g/L. In some cases, methyl-β-cyclodextrin is present in the elicitor composition at a concentration of about 18 g/L.

The elicitor composition may comprise methyl jasmonate (MeJA). Methyl jasmonate is a stress hormone that may activate the biosynthesis of pentacyclic triterpenoids in the hairy root cultures. In some cases, the methyl jasmonate concentration ranges from about 1 μM to about 150 μM, about 50 μM to about 150 μM, or about 100 μM to about 150 μM. In some cases, methyl jasmonate is present in the elicitor composition at a concentration of about 125 M.

The elicitor composition may comprise hydrogen peroxide (H₂O₂). Hydrogen peroxide induces oxidative stress and may also induce ethylene production, which activates the production of pentacyclic triterpenoids in the hairy root cultures. In some cases, the hydrogen peroxide concentration ranges from about 1.5 mM to about 120 mM, about 1.5 mM to about 100 mM, about 1.5 mM to about 80 mM, about 1.5 mM to about 60 mM, about 1.5 mM to about 40 mM, about 1.5 mM to about 20 mM, about 1.5 mM to about 10 mM, or about 2 mM to about 5 mM. In some cases, hydrogen peroxide is present in the elicitor composition at a concentration of about 3 mM.

The elicitor composition may comprise magnesium chloride (MgCl₂). Magnesium chloride may act as a cofactor for enzymes in the biosynthetic pathway, thereby enhancing the production of pentacyclic triterpenoids in the hairy root cultures. In some cases, the magnesium chloride concentration ranges from about 0.5 mM to about 10 mM, 0.5 mM to about 5 mM, about 0.5 to about 4 mM, about 0.5 to about 3 mM, or about 0.5 to about 2 mM. In some cases, magnesium chloride is present in the elicitor composition at a concentration of about 1 mM.

The combination of methyl-β-cyclodextrin, methyl jasmonate, hydrogen peroxide, and magnesium chloride in the elicitor composition may produce synergistic effects on triterpenoid production. The hairy root cultures treated with the combination of all four elicitor components may exhibit enhanced yields of arjunolic acid and 2α,6β-dihydroxybetulinic acid compared to cultures treated with fewer elicitor components. Different combinations of elicitors may selectively enhance the production of different triterpenoids.

The culture medium comprises macronutrients, micronutrients, and vitamins suitable for plant cell culture. Suitably, the culture medium may comprise a carbon source (e.g., as sucrose), a nitrogen source (e.g., ammonium nitrate or potassium nitrate), a phosphate source (e.g., potassium phosphate), a boron source (e.g., boric acid), a mineral or salt source (e.g., calcium chloride, cobalt chloride, cupric sulfate, ferrous sulfate, magnesium sulfate, manganese sulfate, potassium iodide, sodium molybdate, zinc sulfate, or hydrates thereof), a growth factor (e.g., myo-inositol), vitamins (e.g., vitamins B1, B3, or B6), chelators [e.g., ethylenediaminetetraacetic acid (EDTA) or a salt thereof, such as disodium EDTA or ferric sodium EDTA], growth regulators (e.g., auxins or cytokinins), and combinations thereof. An exemplary culture medium is Murashige and Skoog (MSV) medium but others are known in the art. The culture medium used for elicitation may be a modified Murashige and Skoog (MSV) medium supplemented with sucrose. In some cases, the culture medium is supplemented with about 3% sucrose. The culture medium may be maintained at a pH of about 6 prior to elicitation.

The methods disclosed herein comprise extracting pentacyclic triterpenoids from the elicited culture medium and, optionally, from the elicited hairy roots. The extraction process may be performed after the elicitation period to obtain triterpenoid-rich extracts containing the induced pentacyclic triterpenoids.

The pentacyclic triterpenoids may be extracted from the culture medium using organic solvent extraction. In some cases, the culture medium is extracted with ethyl acetate to obtain a triterpenoid-rich extract. The extraction may involve partitioning the aqueous culture medium with ethyl acetate, followed by separation of the organic layer. The organic layer may be dried, filtered, and concentrated to yield a crude extract containing the pentacyclic triterpenoids.

The pentacyclic triterpenoids may also be extracted from the elicited hairy root tissue. The root tissue may be harvested after elicitation, frozen, and lyophilized prior to extraction. The lyophilized root tissue may be extracted using organic solvents to obtain triterpenoids retained within the root tissue. In some cases, the majority of the pentacyclic triterpenoids are secreted into the culture medium during elicitation.

The extraction of pentacyclic triterpenoids from the elicited culture medium may yield a triterpenoid-rich extract containing multiple pentacyclic triterpenoids. In some cases, the triterpenoid-rich extract comprises greater than 50% triterpenoids by weight. The triterpenoid-rich extract may contain arjunolic acid, 2α,6β-dihydroxybetulinic acid, maslinic acid, oleanolic acid, and other pentacyclic triterpenoids in varying proportions. In some cases, the triterpenoid-rich extract contains approximately 35% arjunolic acid, approximately 10% 2α,6β-dihydroxybetulinic acid, approximately 7.5% maslinic acid, and approximately 2% oleanolic acid.

The extraction process may be scaled for larger production volumes. In some cases, the culture medium from multiple flasks of elicited hairy root cultures is pooled and extracted to obtain larger quantities of the triterpenoid-rich extract. The organic extract obtained from the culture medium may be further processed by fractionation and purification techniques to isolate individual pentacyclic triterpenoids.

The methods disclosed herein may include selecting concentrations of methyl-β-cyclodextrin, methyl jasmonate, hydrogen peroxide, and magnesium chloride for the elicitor. These components may be independently selected to enhance the production and secretion of pentacyclic triterpenoids from the hairy root cultures or alter the pentacyclic triterpenoid profile.

In some cases, the methyl-β-cyclodextrin is selected to have a concentration from about 4.5 g/L to about 27 g/L, about 10 g/L to about 25 g/L, or about 15 g/L to about 20 g/L. The concentration of methyl-β-cyclodextrin may be selected to be used in combination with the other elicitor components to achieve enhanced triterpenoid yields.

In some cases, the methyl jasmonate is selected to have a concentration from about 1 μM to about 150 μM, about 50 μM to about 150 μM, or about 100 μM to about 150 μM. The concentration of methyl jasmonate may be selected to be used in combination with the other elicitor components to induce triterpenoid biosynthesis.

In some cases, the hydrogen peroxide is selected to have a concentration from about 1.5 mM to about 120 mM. In some cases, the hydrogen peroxide concentration ranges from about 1.5 mM to about 120 mM, about 1.5 mM to about 100 mM, about 1.5 mM to about 80 mM, about 1.5 mM to about 60 mM, about 1.5 mM to about 40 mM, about 1.5 mM to about 20 mM, about 1.5 mM to about 10 mM, or about 2 mM to about 5 mM. The concentration of hydrogen peroxide may be selected to be used in combination with the other elicitor components to stimulate triterpenoid production.

In some cases, the magnesium chloride is selected to have a concentration from about 1 mM to about 10 mM. In some cases, the magnesium chloride concentration ranges from about 1 mM to about 10 mM, about 1 mM to about 5 mM, or about 1 mM to about 2 mM. The concentration of magnesium chloride may be selected to be used in combination with the other elicitor components to enhance triterpenoid secretion into the culture medium.

The concentrations of the elicitor components were evaluated by treating hairy root cultures with different combinations of elicitors and measuring the resulting triterpenoid yields. The combination of methyl-β-cyclodextrin, methyl jasmonate, hydrogen peroxide, and magnesium chloride produced enhanced yields of arjunolic acid and 2α,6β-dihydroxybetulinic acid compared to other elicitor combinations tested.

Different combinations of elicitor components may selectively enhance the production of different triterpenoids. The combination of methyl-β-cyclodextrin and hydrogen peroxide without methyl jasmonate or magnesium chloride may produce higher yields of maslinic acid and oleanolic acid compared to the four-component elicitor composition. The selection of elicitor component concentrations may be adjusted based on the desired triterpenoid profile in the resulting extract.

In some cases, 2α,6β-dihydroxybetulinic acid is extracted from the elicited culture medium. The 2α,6β-dihydroxybetulinic acid is a lupane-type triterpenoid that may be induced upon elicitation with the elicitor composition. In some cases, 2α,6β-dihydroxybetulinic acid is not detected in the culture medium of non-elicited control hairy root cultures, indicating that elicitation induces the production and secretion of 2α,6β-dihydroxybetulinic acid.

In some cases, maslinic acid is extracted from the elicited culture medium. Maslinic acid is an oleanane-type triterpenoid that may be induced upon elicitation. In some cases, maslinic acid is not detected in the culture medium of non-elicited control hairy root cultures. The yield of maslinic acid in the elicited culture medium may vary depending on the elicitor combination used.

In some cases, oleanolic acid is extracted from the elicited culture medium. Oleanolic acid is an oleanane-type triterpenoid that may be induced upon elicitation. In some cases, oleanolic acid is not detected in the culture medium of non-elicited control hairy root cultures. The yield of oleanolic acid in the elicited culture medium may vary depending on the elicitor combination used.

In some cases, 6β-hydroxybetulinic acid is extracted from the elicited culture medium. The 6β-hydroxybetulinic acid is a lupane-type triterpenoid that may be present in the elicited culture medium. The yield of 6β-hydroxybetulinic acid in the elicited culture medium may vary depending on the elicitor combination used.

In some cases, arjunolic acid is extracted from the elicited culture medium. Arjunolic acid is an oleanane-type triterpenoid that may be present in both elicited and non-elicited hairy root cultures. The yield of arjunolic acid in the elicited culture medium may be higher compared to non-elicited control cultures. Arjunolic acid may be the predominant triterpenoid in the elicited culture medium. The yield of arjunolic acid in the elicited culture medium may vary depending on the elicitor combination used.

In some cases, betulinic acid is extracted from the elicited culture medium. Betulinic acid is a lupane-type triterpenoid that may be present in the elicited culture medium. The yield of betulinic acid in the elicited culture medium may vary depending on the elicitor combination used.

In some cases, alphitolic acid is extracted from the elicited culture medium. Alphitolic acid is a lupane-type triterpenoid that may be present in the elicited culture medium. The yield of alphitolic acid in the elicited culture medium may vary depending on the elicitor combination used.

The extraction of multiple pentacyclic triterpenoids from the elicited culture medium may provide a triterpenoid-rich extract containing a mixture of oleanane-type and lupane-type triterpenoids with varying bioactivity profiles. In some instances, the combination of pentacyclic triterpenoid may demonstrate synergistic activity compared to individual pentacyclic triterpenoids at equivalent amounts, e.g., molar equivalent amounts.

The methods disclosed herein comprise eliciting the hairy roots for a defined time period to induce the production and secretion of pentacyclic triterpenoids. In some cases, eliciting the hairy roots comprises eliciting for between 24 hours and 240 hours, e.g., between 48 hours and 192 hours. In some cases, eliciting the hairy roots comprises eliciting for between 48 hours and 96 hours, between 96 hours and 144 hours, or between 144 hours and 192 hours. The elicitation duration may be selected based on the desired triterpenoid yield and the specific triterpenoid profile to be obtained from the elicited culture medium.

The accumulation of pentacyclic triterpenoids in the culture medium may increase over the elicitation time period. The elicitation time period may affect the yields of different pentacyclic triterpenoids to varying degrees. The selection of the elicitation time period may be adjusted based on the target triterpenoid or triterpenoid profile to be obtained. The elicitation time period may be selected in combination with the elicitor composition and culture medium to achieve the desired triterpenoid production profile.

The methods disclosed herein may further comprise re-eliciting the hairy roots with the elicitor composition after extracting triterpenoids. The re-elicitation process involves treating the previously elicited hairy root cultures with the elicitor composition comprising methyl-β-cyclodextrin, methyl jasmonate, hydrogen peroxide, and magnesium chloride for an additional elicitation period. The re-elicitation process demonstrates the capacity of the hairy root system to respond to multiple elicitor treatments and produce additional pentacyclic triterpenoids. The re-elicited cultures may produce additional quantities of pentacyclic triterpenoids that accumulate in the culture medium and root tissue.

The yields of individual pentacyclic triterpenoids obtained from re-elicited cultures may be lower than the yields obtained from the first elicitation. The combined yields from re-elicited culture medium and root tissue may contribute to the total triterpenoid production from the hairy root culture system.

The re-elicitation strategy may be employed for sustainable production of pentacyclic triterpenoids from Bixa orellana hairy root cultures. The hairy roots may be subjected to multiple cycles of elicitation and extraction to maximize the total triterpenoid yield from the culture system. The re-elicitation process may extend the productive lifespan of the hairy root cultures and increase the efficiency of triterpenoid production.

The methods disclosed herein may further comprise culturing the hairy roots in the culture medium for a period prior to elicitation. In some cases, the hairy roots are cultured in the culture medium for between about 10 days and about 20 days before elicitation. In some cases, the hairy roots are cultured in the culture medium for between about 10 days and 15 days or about 12 days and about 15 days before elicitation. In some cases, the hairy roots are cultured in the culture medium for about 12 days before elicitation. The pre-elicitation culture period allows the hairy roots to establish and reach a growth phase suitable for elicitation.

The timing of elicitation may be selected based on the growth phase of the hairy root cultures. The hairy roots of Bixa orellana may exhibit a lag phase during the initial days of culture, followed by an exponential growth phase, and subsequently a stationary phase. The pre-elicitation culture period may be selected to coincide with the exponential growth phase of the hairy roots.

Culturing the hairy roots for about 12 days before elicitation may position the cultures within the exponential growth phase. The hairy roots in the exponential growth phase may exhibit active metabolic activity and may be responsive to elicitor treatment. The selection of the pre-elicitation culture period may influence the capacity of the hairy roots to produce and secrete pentacyclic triterpenoids upon elicitation.

The culture medium used during the pre-elicitation culture period may be the same as or different from the culture medium used during elicitation. As described previously, the culture medium may be a modified Murashige and Skoog medium supplemented with sucrose. The hairy roots may be maintained in the culture medium under conditions that support growth.

The pre-elicitation culture period may be monitored by measuring parameters, such as pH and conductivity of the culture medium. Changes in pH and conductivity may indicate nutrient uptake by the hairy roots and progression through the growth phases. The pH of the culture medium may decrease during the initial days of culture and then gradually increase during the remaining growth period. The conductivity of the culture medium may decrease as the hairy roots utilize nutrients from the medium.

The methods disclosed herein may further comprise purifying the extracted triterpenoids. The purification process may involve fractionation and isolation techniques to obtain individual pentacyclic triterpenoids from the triterpenoid-rich extract. The purification of extracted triterpenoids may be performed using column chromatography and semi-preparative high-performance liquid chromatography (HPLC) techniques.

The triterpenoid-rich extract obtained from the elicited culture medium may be fractionated by column chromatography, e.g., silica gel column chromatography. The silica gel column chromatography may employ a gradient of of solvents as the mobile phase. In some cases, the gradient comprises chloroform:methanol ratios of 1:0, 20:1, 10:1, 4:1, 2:1, 1:1, and 0:1 (v/v). The fractions obtained from silica gel column chromatography may be concentrated and further processed to isolate individual pentacyclic triterpenoids.

The fractions obtained from silica gel column chromatography may be further fractionated by reversed-phase octadecylsilyl (ODS) column chromatography. The ODS column chromatography may employ a gradient of acetonitrile and 0.5% formic acid as the mobile phase. In some cases, the gradient comprises acetonitrile: 0.5% formic acid ratios of 2:8, 3:7, 4:6, 5:5, 6:4, 7:3, 8:2, and methanol (v/v). The fractions obtained from ODS column chromatography may be concentrated and extracted with ethyl acetate, and the organic layer may be dried over anhydrous sodium sulfate, filtered, and concentrated to yield semi-pure fractions containing pentacyclic triterpenoids.

The semi-pure fractions obtained from column chromatography may be purified by semi-preparative HPLC. The semi-preparative HPLC purification may employ a reversed-phase C-18 column. Separation of arjunolic acid, 2α,6β-dihydroxybetulinic acid, and maslinic acid demonstrates the resolution achieved by the semi-preparative HPLC purification method. The distinct retention times of the individual triterpenoids allow for collection of purified fractions containing each compound.

The purity of the isolated pentacyclic triterpenoids may be confirmed by NMR spectroscopy. The structures of the purified triterpenoids may be elucidated by comparing the obtained MS and NMR spectra with previous reports. The purification process may yield individual pentacyclic triterpenoids suitable for characterization and bioactivity evaluation.

The culture medium used in the methods disclosed herein may be Murashige and Skoog medium supplemented with sucrose. The Murashige and Skoog medium, also referred to as MS medium, is a plant tissue culture medium that provides macro- and micronutrients to support plant cell and tissue growth. The modified Murashige and Skoog medium, designated as MSV medium, may be employed for culturing hairy roots of Bixa orellana.

The MSV medium may be supplemented with sucrose. In some cases, the MSV medium is supplemented with about 3% sucrose. The sucrose provides energy and carbon for metabolic processes in the hairy roots, including the biosynthesis of pentacyclic triterpenoids. The concentration of sucrose in the culture medium may influence the growth rate and metabolic activity of the hairy root cultures.

The MSV medium may be prepared as a liquid medium for culturing hairy roots in suspension. The liquid MSV medium allows for nutrient exchange between the culture medium and the hairy root tissue. The hairy roots may be cultured in flasks containing the liquid MSV medium on an orbital shaker incubator to provide agitation and aeration.

The pH of the MSV medium may be adjusted prior to culturing the hairy roots. In some cases, the MSV medium is adjusted to a pH of about 6. The pH of the culture medium may change during the culture period as the hairy roots take up nutrients and release metabolites into the medium. Monitoring the pH of the culture medium may provide information about the metabolic state of the hairy root cultures.

The MSV medium supplemented with sucrose supports the growth of Bixa orellana hairy roots through the lag phase, exponential growth phase, and stationary phase. The composition of the culture medium may influence the biomass accumulation and the capacity of the hairy roots to respond to elicitation. The hairy roots cultured in MSV medium supplemented with sucrose may achieve biomass levels suitable for elicitation and triterpenoid production.

During elicitation, the spent MSV medium may be replaced with fresh MSV medium containing the elicitor composition comprising methyl-β-cyclodextrin, methyl jasmonate, hydrogen peroxide, and magnesium chloride. The fresh MSV medium supplemented with sucrose and the elicitor components provides the nutritional and elicitation conditions for inducing pentacyclic triterpenoid production and secretion into the culture medium.

The distribution of pentacyclic triterpenoids between the culture medium and root tissue demonstrates that the majority of the induced triterpenoids are secreted into the culture medium during elicitation. The secretion of pentacyclic triterpenoids into the culture medium may facilitate downstream extraction and purification processes by reducing the need for extensive root tissue processing.

The comparison between elicited and non-elicited hairy root cultures demonstrates that elicitation with the elicitor composition substantially enhances pentacyclic triterpenoid production in B. orellana hairy roots.

The compositions disclosed herein comprise cultured hairy roots of Bixa orellana in a culture medium, an elicitor comprising methyl-β-cyclodextrin, methyl jasmonate, hydrogen peroxide, and magnesium chloride, and pentacyclic triterpenoids. The pentacyclic triterpenoids may comprise 2α,6β-dihydroxybetulinic acid, maslinic acid, oleanolic acid, and 6β-hydroxybetulinic acid. These pentacyclic triterpenoids may be present in the culture medium as a result of secretion from the elicited hairy roots. The pentacyclic triterpenoids may also be present within the hairy root tissue.

In some cases, the pentacyclic triterpenoids in the composition further comprise arjunolic acid. Arjunolic acid is an oleanane-type triterpenoid that may be present in both elicited and non-elicited hairy root cultures, as described previously. The yield of arjunolic acid may be enhanced upon elicitation with the elicitor composition.

In some cases, the pentacyclic triterpenoids in the composition further comprise betulinic acid, alphitolic acid, or a combination thereof. Betulinic acid and alphitolic acid are lupane-type triterpenoids that may be present in the elicited culture medium. The composition may contain a mixture of oleanane-type and lupane-type pentacyclic triterpenoids with varying bioactivity profiles.

The composition may be prepared by the methods disclosed herein, comprising eliciting cultured hairy roots of Bixa orellana in a culture medium with the elicitor. The composition represents an intermediate stage in the production of pentacyclic triterpenoids, wherein the hairy roots have been elicited and the pentacyclic triterpenoids have accumulated in the culture medium and root tissue prior to extraction.

The compositions disclosed herein comprise a triterpenoid-rich extract obtained from elicited cultured hairy roots of Bixa orellana in a culture medium. The triterpenoid-rich extract may be obtained by extracting the elicited culture medium with a solvent. The triterpenoid-rich extract comprises 2α,6β-dihydroxybetulinic acid, maslinic acid, oleanolic acid, and 6β-hydroxybetulinic acid.

The triterpenoid-rich extract may contain the pentacyclic triterpenoids in varying proportions depending on the elicitation conditions and extraction procedures employed. In some cases, the triterpenoid-rich extract comprises greater than 50% triterpenoids by weight. The triterpenoid-rich extract may be obtained as a crude extract or may be subjected to further purification to enrich the pentacyclic triterpenoid content.

In some cases, the triterpenoid-rich extract further comprises arjunolic acid. As described previously, arjunolic acid may be the predominant triterpenoid in the elicited culture medium.

In some cases, the triterpenoid-rich extract further comprises betulinic acid, alphitolic acid, or a combination thereof.

The triterpenoid-rich extract containing multiple pentacyclic triterpenoids may exhibit combined bioactivities attributable to the individual triterpenoid components.

The triterpenoid-rich extract may be prepared by the methods disclosed herein, comprising eliciting cultured hairy roots of Bixa orellana with the elicitor composition and extracting pentacyclic triterpenoids from the elicited culture medium. The triterpenoid-rich extract represents a processed form of the pentacyclic triterpenoids produced by the elicited hairy root cultures.

The triterpenoid-rich extract may further comprise a pharmaceutically acceptable carrier, excipient, or diluent. The pharmaceutically acceptable carrier, excipient, or diluent may be selected based on the intended route of administration and formulation requirements. The triterpenoid-rich extract formulated with a pharmaceutically acceptable carrier, excipient, or diluent may be suitable for therapeutic applications.

The methods disclosed herein may further comprise derivatives of the pentacyclic triterpenoids obtained from the elicited hairy root cultures. For example, one or more pentacyclic triterpenoids may be acetylated.

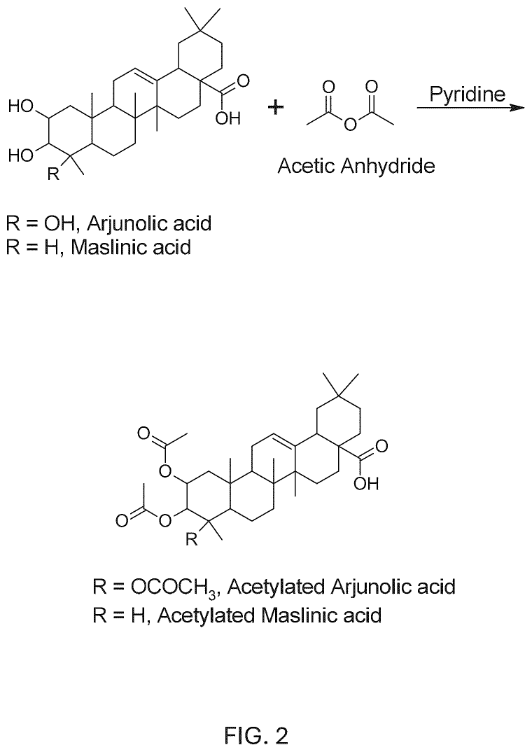

Referring to FIG. 2, a reaction schematic depicts the semi-synthesis of acetylated derivatives of arjunolic acid and maslinic acid using acetic anhydride and pyridine. The reaction involves treating the pentacyclic triterpenoid starting material with acetic anhydride in the presence of pyridine under anhydrous conditions. The hydroxyl groups present on the triterpenoid backbone undergo acetylation to form acetate ester groups.

The methods disclosed herein comprise inhibiting induction of an inflammatory marker. The method comprises contacting an immune cell with the composition comprising a triterpenoid-rich extract obtained from elicited cultured hairy roots of Bixa orellana or a pentacyclic triterpenoid obtained from the composition. The triterpenoid-rich extract may further comprise a pharmaceutically acceptable carrier, excipient, or diluent.

In some cases, the immune cell is a macrophage, such as RAW 264.7 macrophages used in the Examples. The macrophage may be stimulated with an inflammatory stimulus, such as lipopolysaccharide (LPS), to induce production of inflammatory markers. The triterpenoid-rich extract or pentacyclic triterpenoid may be contacted with the macrophage prior to, concurrently with, or after exposure to the inflammatory stimulus.

In some cases, the inflammatory marker is nitrite. Nitrite production by macrophages may serve as an indicator of nitric oxide production and inflammatory response. The triterpenoid-rich extract or pentacyclic triterpenoid may reduce nitrite levels in stimulated macrophages compared to macrophages treated with the inflammatory stimulus alone.

The pentacyclic triterpenoids obtained from the composition may exhibit varying degrees of anti-inflammatory activity. In some cases, acetylated derivatives demonstrate enhanced reduction in nitrite levels compared to the parent compounds.

The triterpenoid-rich extract may demonstrate anti-inflammatory activity that differs from the activity of individual pentacyclic triterpenoids at molar equivalent concentrations. In some cases, the triterpenoid-rich extract exhibits greater reduction in nitrite levels compared to individual pentacyclic triterpenoids tested at molar equivalent concentrations, suggesting a combined or synergistic effect of the multiple pentacyclic triterpenoids present in the extract.

The method of inhibiting induction of an inflammatory marker may be performed by pre-treating the immune cell with the triterpenoid-rich extract or pentacyclic triterpenoid for a period prior to exposure to the inflammatory stimulus. In some cases, the immune cell is pre-treated for about 2 hours before exposure to the inflammatory stimulus. The immune cell may then be co-incubated with the inflammatory stimulus for a period, such as about 24 hours, before measuring the inflammatory marker.

The methods disclosed herein comprise inhibiting growth or proliferation of bacteria. The method comprises contacting the bacteria with the composition comprising a triterpenoid-rich extract obtained from elicited cultured hairy roots of Bixa orellana or a pentacyclic triterpenoid obtained from the composition. As described previously, the triterpenoid-rich extract may further comprise a pharmaceutically acceptable carrier, excipient, or diluent.

In some cases, the bacteria is Gram-positive or Gram-negative. Exemplary Gram-positive bacteria include, without limitation, Bacillus subtilis, Enterococcus faecalis, Enterococcus faecium, Staphylococcus aureus, and Streptococcus mutans. Exemplary Gram-negative bacteria include, without limitation, Escherichia coli and Klebsiella pneumoniae.

In some cases, the bacteria is resistant to one or more antibiotics. For example, the bacteria may be methicillin or vancomycin resistant, such as Methicillin-resistant S. aureus (MRSA), vancomycin-resistant E. faecium, or vancomycin-resistant E. faecalis.

The antibacterial activity of the pentacyclic triterpenoids may vary depending on the structural type of the triterpenoid. Oleanane-type triterpenoids, such as maslinic acid and oleanolic acid, may exhibit higher antibacterial activity compared to lupane-type triterpenoids against certain bacterial strains. The number and position of hydroxyl groups on the triterpenoid backbone may influence the antibacterial activity profile.

The method of inhibiting growth or proliferation of bacteria may be performed by contacting the bacteria with the triterpenoid-rich extract or pentacyclic triterpenoid at concentrations sufficient to inhibit bacterial growth. The MIC refers to the concentration at which microbial growth is inhibited. The triterpenoid-rich extract or pentacyclic triterpenoid may be contacted with the bacteria in a liquid culture medium or other suitable environment for assessing antibacterial activity.

The methods disclosed herein comprise inhibiting growth or proliferation of cancer cells. The method comprises contacting the cancer cells with the composition comprising a triterpenoid-rich extract obtained from elicited cultured hairy roots of Bixa orellana or a pentacyclic triterpenoid obtained from the composition. As described previously, the triterpenoid-rich extract may further comprise a pharmaceutically acceptable carrier, excipient, or diluent.

In some cases, the cancer cells are breast cancer cells, such as MCF-7 cells. The breast cancer cells may be MCF-7 cells. MCF-7 cells are a human breast adenocarcinoma cell line that may be used to evaluate the cytotoxic effects of pentacyclic triterpenoids and triterpenoid-rich extracts.

The method of inhibiting growth or proliferation of cancer cells may be performed by contacting the cancer cells with the triterpenoid-rich extract or pentacyclic triterpenoid for a defined treatment period. In some cases, the cancer cells are contacted with the triterpenoid-rich extract or pentacyclic triterpenoid for about 72 hours. The treatment period may be selected based on the desired cytotoxic effect and the specific cancer cell type being treated.

The pentacyclic triterpenoids obtained from the composition may exhibit varying degrees of cytotoxic activity against cancer cells. The IC₅₀ values, representing the concentration at which 50% cell viability is observed, may vary among the different pentacyclic triterpenoids. The structural type of the triterpenoid and the number and position of functional groups on the triterpenoid backbone may influence the cytotoxic activity profile against cancer cells.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Materials and Methods

General Experimental Procedures. The NMR spectra of the purified and synthesized terpenoids were obtained using a JEOL JNM-ECZ400s spectrometer (JEOL USA, MA, USA) at 400 MHz for ¹H and 101 MHz for ¹³C in DMSO-d₆ using the signals of the residual solvent (δ_{H} 2.50) and (δ_{C} 39.5) as internal standards and CDCl₃ using residual solvent signals (δ_{H} 7.26) and (δ_{C} 77.2). Mass spectrometry data was obtained using LTQ XL linear ion trap mass spectrometer (Thermo Scientific, Waltham, MA, USA) with an electrospray ionization (ESI) source following the previously described protocol.²²

Hairy Root Cultures of B. orellana. The previously established B. orellana hairy root line 15 was maintained in 500 mL flasks with 100 mL of modified liquid Murashige and Skoog (MSV) medium²³ supplemented with 3% sucrose at pH 6.^{12,24} The cultures were maintained in an orbital shaker incubator with conditions described previously.²⁵

Growth Kinetics of Hairy Roots. A growth curve was established following a previously described procedure.^{23,25} Briefly, twelve 2-3 cm tips of B. orellana hairy roots were cultured in 500 mL flasks containing 100 mL of MSV medium with 3% sucrose to establish a growth curve. Three flasks were harvested every three days for 30 days, and then the roots were frozen, followed by lyophilization (Freeze Dry System Freezone 4.5, Labconco™, Kansas City, MO, USA). The specific growth rate, doubling time, and growth index of the lyophilized hairy roots were calculated as previously described.^{23,25} The pH and conductivity of the culture medium were measured at each time point.

Elicitation of Hairy Root Cultures. Four combinations of elicitors in MSV medium containing 3% sucrose were tested as follows:
18 g/L CD (CAVASOL® W7 M, Wacker, Munich, Germany)+125 μM MeJA (Sigma-Aldrich, St. Louis, MO, USA).²⁶
18 g/L CD+3 mM H₂O₂ (Thermo Scientific, Waltham, MA, USA)
18 g/L CD+125 μM MeJA+3 mM H₂O₂
18 g/L CD+125 μM MeJA+3 mM H₂O₂+1 mM MgCl₂ (Sigma-Aldrich, St. Louis, MO, USA)
50 μL absolute ethanol (solvent of MeJA; Control)

The hairy roots were cultured for 12 days in 500 mL flasks containing 100 mL of MSV medium before elicitation. Elicitation was then conducted for 192 h in continuous darkness, with the spent medium replaced with 200 mL of MVS medium containing the various elicitor combinations, as described above. Elicitation was performed in triplicate under the conditions outlined before.²⁶ For HPLC analysis, 3 mL aliquots were taken from the elicited cultures at 48, 96, 144, and 192 h.

Re-elicitation of Hairy Root Cultures. To determine the capacity of the hairy roots to produce triterpenoids upon re-elicitation, the elicited hairy roots cultures were re-treated with MSV medium containing 125 μM MeJA, 1 mM MgCl₂, 3 mM H₂O₂, and 18 g/L CD for 192 h.²⁶ The roots were maintained in the conditions described previously. The experiments were performed in triplicate and included control groups, as mentioned above. Aliquots collected from the medium of the re-elicited hairy root cultures were analyzed using HPLC following the procedure described below.

Extraction and Analysis of Triterpenoids from Hairy Root Cultures. To extract the triterpenoids from the culture medium and root tissue, a previously established protocol was used.²⁷ The triterpenoid-rich extract from the elicited culture medium and root tissue was analyzed using HPLC. HPLC analysis was performed using a Sunfire™ C18 column (4.6×250 mm, 5 μm, Waters, Milford, MA, USA) at a flow rate of 1.0 mL/min and maintained at 40° C. The mobile phase consisted of water (A) and acetonitrile (MeCN) (B) (v/v). Analytical HPLC was used to perform separation using an acetonitrile gradient: 15% B to 85% B from 0 to 30 min and 85% B to 100% B from 30 to 35 min. The same method was used for the analysis of re-elicited hairy root media and root tissue.

Calibration curves were generated using several dilutions of purified triterpenoids in methanol. The purified triterpenoids were purified as described below. The calibration curves were established using absorbance at 200 nm for arjunolic acid (y=0.1274x+0.5689, LOQ=42.05 mg/L, LOD=13.87 mg/L, R²=0.999), 2α,6β-dihydroxybetulinic acid (y=0.1219x+0.8176, LOQ=63.26 mg/L, LOD=20.88 mg/L, R²=0.9998), maslinic acid (y=0.1271x+0.5938, LOQ=96.16 mg/L, LOD=31.73 mg/L, R²=0.996), and oleanolic acid (y=0.127x+1.5614, LOQ=191.331 mg/L, LOD=63.14 mg/L, R²=0.9988).

Purification of Triterpenoids from Elicited Hairy Root Cultures. A total of 12-14 B. orellana hairy root tips were cultured in each of twenty-five 500 mL flasks containing 100 mL of MSV medium supplemented with 3% sucrose. After 12 d, the medium was replaced with fresh MSV medium containing 125 μM MeJA, 1 mM MgCl₂, 3 mM H₂O₂, 18 g/L CD, and 3% sucrose to start the elicitation process. The elicited cultures were maintained in continuous darkness for 192 h. An organic extract of 3.49 g was obtained from 5 L of the elicited culture medium following a previously described protocol.²⁵ The culture medium was partitioned with ethyl acetate, and the ethyl acetate phase containing the triterpenoids was collected to provide the extract.

The extract was then fractionated by silica gel column chromatography (Sigma-Aldrich, St. Louis, MO, USA) using a gradient of chloroform:methanol (1:0, 20:1, 10:1, 4:1, 2:1, 1:1, and 0:1, v/v) and reversed-phase octadecylsilyl (ODS) column chromatography (Nacalai Tesque, Inc., Kyoto, Japan), following previously described methods, to isolate purified triterpenoids.²⁵ The fraction eluted with a 10:1 chloroform-methanol ratio was concentrated to 1.43 g of pale brown oil, which was further fractionated by ODS column chromatography with a gradient of acetonitrile-0.5% formic acid (2:8, 3:7, 4:6, 5:5, 6:4, 7:3, 8:2, and MeOH v/v). Fraction 8 (MeOH) was concentrated and extracted with ethyl acetate, and the organic layer was dried over anhydrous Na₂SO₄, filtered, and concentrated to yield a 396.8 mg semi-pure fraction. Fractions 5 and 6 eluted with 4:1 and 2:1 chloroform-methanol ratios from silica gel column chromatography were concentrated to yield a total of 1.79 g of pale brown oil.

Finally, the combined silica gel fractions (4:1 and 2:1) were purified by semi-preparative HPLC (SunFire C18 OBD™ Prep, Waters, Milford, MA, USA; 10×250 mm, 4 mL/min, UV detection at 200 nm). For this combined fraction, gradient elution of MeCN/water solution (15:85 to 85:15 for 30 min, 85:15 to 100:0 over 30 min) and yielded 232 mg of arjunolic acid, 58 mg of 2α,6β-dihydroxybetulinic acid, and 10 mg of maslinic acid (Figure S1). Similarly, fraction 8, obtained after ODS column chromatography, was purified via gradient elution with MeCN/water solution (55:45 to 90:10 for 30 min, then 90:10 to 100:0 over 30 min) yielded 13.5 mg of alphitolic acid, 131 mg of maslinic acid, 8.2 mg of 6β-hydroxybetulinic acid, 8.1 mg of betulinic acid, and 40.6 mg of oleanolic acid (Figure S2). The purity of these compounds was checked by NMR.

Synthesis of Acetylated Derivatives of Arjunolic Acid and Maslinic Acid. Arjunolic acid (55 mg, 0.11 mmol), pyridine (0.5 mL), and acetic anhydride (1 mL) were combined (FIG. 2). The reaction mixture was stirred and maintained at room temperature overnight under anhydrous conditions. Subsequently, 10% HCl was added to quench the reaction, and the resulting precipitate was filtered and thoroughly washed with distilled water. The desired product was then vacuum-dried to yield tri-acetylated derivative of arjunolic acid as a white powder (53 mg, 0.086 mmol, 76.78%). ¹H NMR (400 MHz, CDCl₃) δ 5.27 (t, J=3.34 Hz), 5.14 (td, J=10.8, 4.4 Hz), 5.07 (d, J=10.8 Hz), 3.82 (d, J=11.8 Hz), 3.56 (d, J=11.8 Hz), 2.81 (dd, J=13.9, 4.4 Hz), 2.07 (s, 3H,), 2.02 (s, 3H), 1.99 (s, 3H), 1.12 (s, 3H), 1.06 (s, 3H), 0.91 (s, 3H), 0.89 (s, 3H), 0.86 (s, 3H), 0.72 (s, 3H). ¹³C NMR (101 MHz, CDCl₃): δ 183.5, 170.9, 170.6, 170.5, 143.7, 122.1, 77.3, 69.9, 65.3, 47.7, 47.5, 46.5, 45.8, 43.5, 41.9, 41.6, 40.9, 39.3, 37.9, 33.8, 33.1, 32.4, 32.1, 30.7, 27.6, 25.8, 23.6, 23.5, 22.8, 21.2, 21.0, 20.8, 17.9, 17.0, 16.9, 13.9. ESI-MS m/z: 613.52 [M−H]⁻ (calcd for C₃₆H₅₃O₈⁻, 613.3745).

Similarly, maslinic acid (55 mg, 0.11 mmol) was subjected to the same procedure to yield 45 mg acetylated derivative as a white powder (45 mg, 0.081 mmol; 69.82%). ¹H NMR (400 MHz, DMSO-d₆): δ 5.11 (t, J=3.34 Hz), 4.93 (td, J=10.8, 4.4 Hz), 4.62 (d, J=10.8 Hz), 2.70 (dd, J=13.9, 4.4 Hz), 1.97 (s, 3H), 1.88 (s, 3H), 1.07 (s, 3H), 0.94 (s, 3H), 0.83 (s, 3H), 0.83 (s, 3H), 0.80 (s, 3H), 0.78 (s, 3H), 0.67 (s, 3H). ¹³C NMR (101 MHz, DMSO-d₆): δ 179.1, 170.5, 170.3, 144.4, 121.6, 80.1, 79.7, 69.6, 54.2, 47.0, 46.1, 45.9, 43.6, 41.9, 41.2, 39.4, 38.2, 33.8, 33.3, 32.6, 32.5, 30.9, 28.5, 27.7, 26.1, 23.8, 23.4, 23.1, 21.3, 21.1, 18.3, 18.0, 17.2, 16.6. ESI-MS m/z: 555.61 [M−H]⁻ (calcd for C₃₄H₅₁O₆, 555.3691).

Cell Culture. RAW 264.7 mouse macrophages (ATCC® TIB-71) were obtained from the American Type Culture Collection (ATCC; Manassas, VA, USA) and maintained in Dulbecco's Modified Eagle's medium (DMEM) supplemented with 100 μg/mL streptomycin, 100 U/mL penicillin (Gibco; Life Technologies, Grand Island, NY, USA), and 10% fetal bovine serum (FBS; HyClone, Massachusetts, USA) at 37° C. in a humidified incubator with 5% CO₂. Triterpenoids were dissolved in dimethyl sulfoxide (DMSO; ATCC; Manassas, VA, USA) for in vitro assays.

Cytotoxicity Assay. The cytotoxic effects of the triterpenoids on RAW 264.7 macrophages were determined by the method suggested by the manufacturer using the RealTime-Glo™ MT Cell Viability Assay (Promega, WI, USA). A total of 4000 cells/well were seeded into a clear-bottom 96-well plate and cultured overnight. Afterward, the culture medium was replaced with various concentrations of the triterpenoids from 3.12 to 100 μM and with RealTime-Glo™ MT Cell Viability Assay reagents. Cells treated with 0.05% DMSO were used as controls. Luminescence was read after 24 h using a BioTek Synergy HTX reader (Agilent Technologies, Santa Clara, CA, USA).

Nitrite Measurement. Nitrite levels were measured 24 h after lipopolysaccharide (LPS) stimulation. Briefly, RAW 264.7 cells were cultured overnight in 24-well plates at 10⁵ cells/well. The cells were pre-treated for 2 h with the triterpenoids at concentrations of 20 μM and 35 μM, followed by 24-h co-stimulation with LPS (1 μg/mL). Following incubation, the culture medium was collected, and nitrite levels were determined using the Griess Reagent System (Promega, WI, USA), according to the manufacturer's instructions.

Antimicrobial Assay. Minimum inhibitory concentration (MIC) was determined using liquid microcultures in 96-well microtiter plates. The triterpenoids were tested against Bacillus subtilis ATCC 6623, Escherichia coli ATCC 25922, Enterococcus faecalis ATCC 29212, E. faecalis ATCC 51299 (VRE), Enterococcus. faecium ATCC 700221 (VRE), Klebsiella pneumoniae ATCC 700603, Staphylococcus aureus ATCC 25923, S. aureus ATCC 35591 (MRSA), S. aureus ATCC 00699 (MRSA), and Streptococcus mutans UA159. All the strains were cultured under aerobic conditions at a temperature of 37° C., except for S. mutans, which was cultured at 37° C. under 5% CO₂. Cation-adjusted Muller-Hinton Broth (CAMHB; Difco) was used as the medium in the antimicrobial assay, except for S. mutans, for which Brain Heart Infusion broth (BHI; Difco) was used as S. mutans does not grow in CAHMB.

All the compounds, including arjunolic acid, 2α,6β-dihydroxybetulinic acid, maslinic acid, oleanolic acid, betulinic acid, 6β-hydroxybetulinic acid, alphitolic acid, and the two semi-synthesized compounds, were screened against the bacterial strains at concentrations ≤100 μg/mL. Next, the compounds were 2-fold diluted down the columns of the 96-well plates using CAMHB media, followed by inoculating test bacteria to each well at 10⁶ CFU/mL. For S. mutans, BHI medium was used as the diluent. The plates were incubated at 37° C. for 24 h to determine the concentration at which the microbial growth was completely inhibited. Vancomycin hydrochloride in H₂O was used as the positive control for Gram-positive strains, daptomycin in H₂O was used for vancomycin-resistant enterococci (VRE), and colistin sulfate in H₂O was used for Gram-negative strains. Bacterial strains treated with 0.1% DMSO served as negative controls. Minimum inhibitory concentration (MIC) refers to the concentration at which microbial growth is completely inhibited.

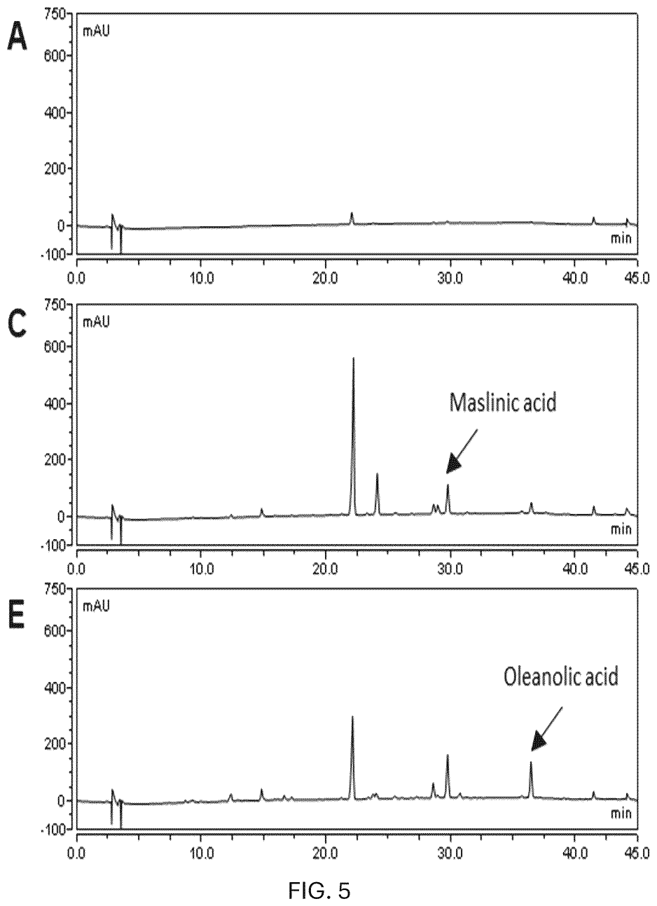

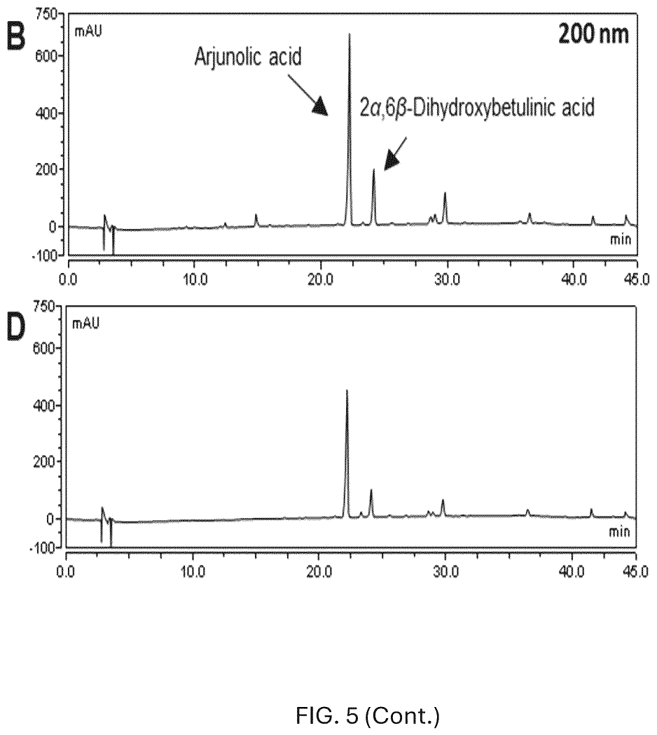

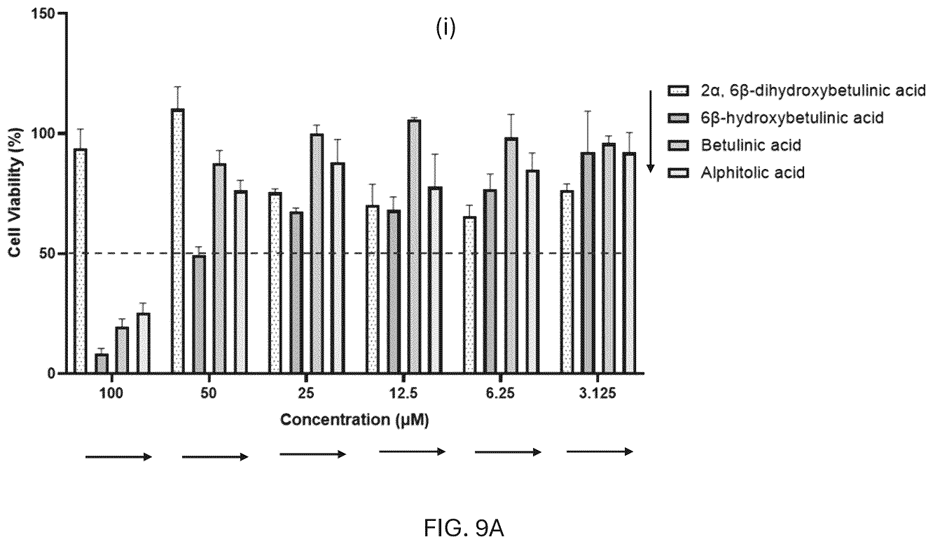

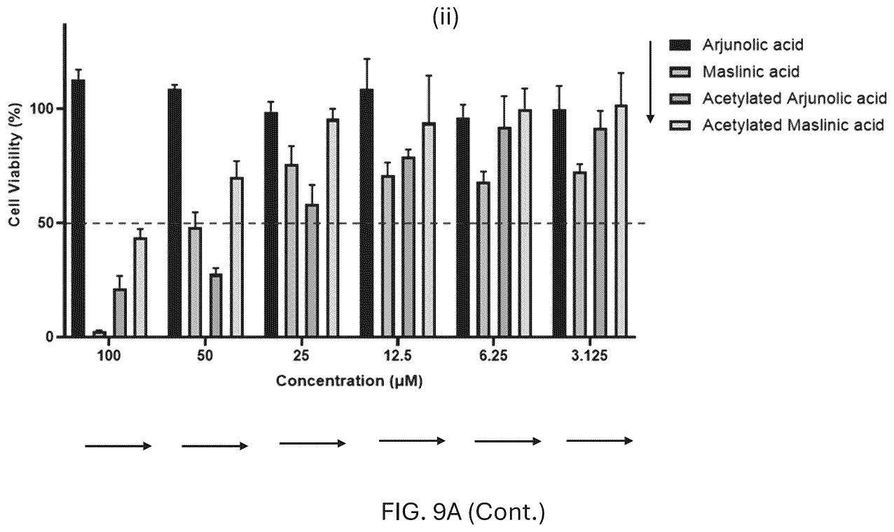

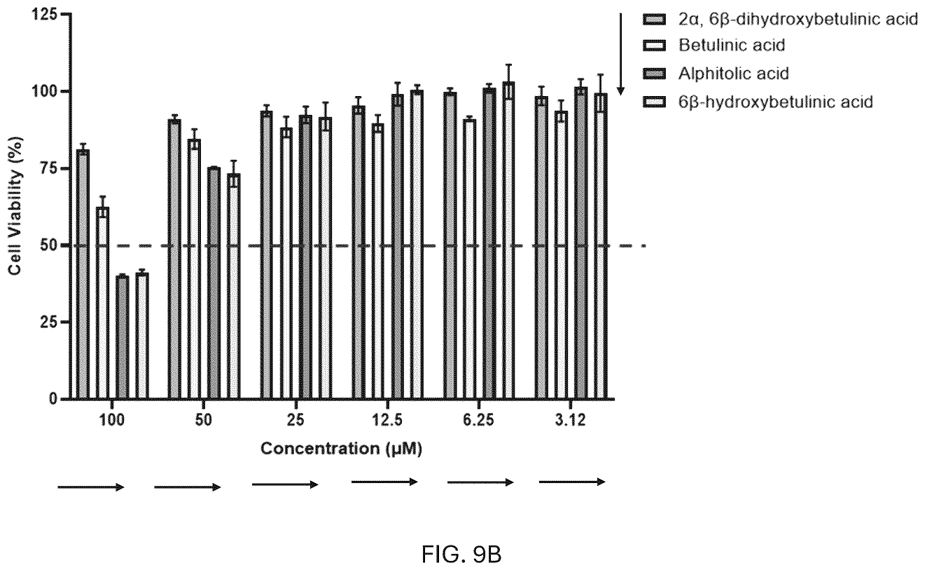

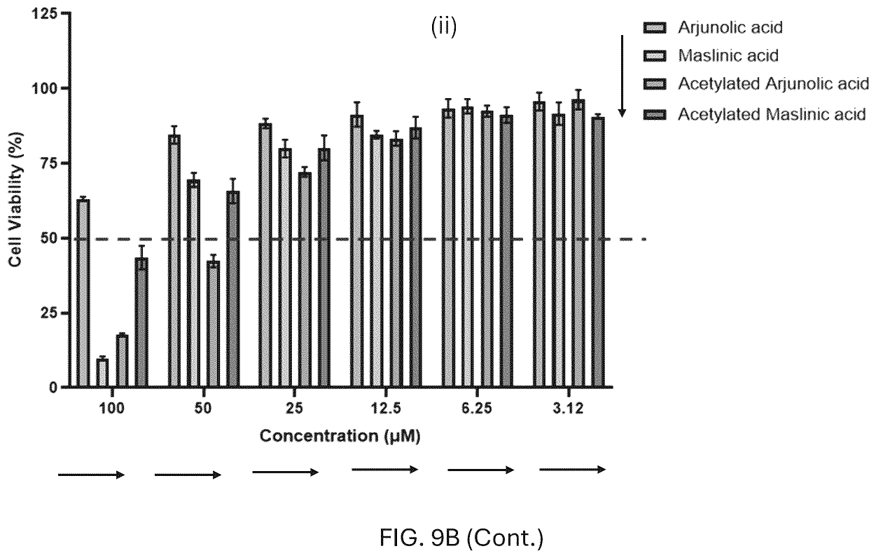

Statistical Analysis. For FIG. 5, a two-way ANOVA followed by Tukey's multiple comparison test was performed, whereas, for FIG. 9, a Brown-Forsythe one-way ANOVA with Dunnett's post-test was applied. All statistical analyses were conducted using the GraphPad Prism 9 software (San Diego, CA, USA).

### Results

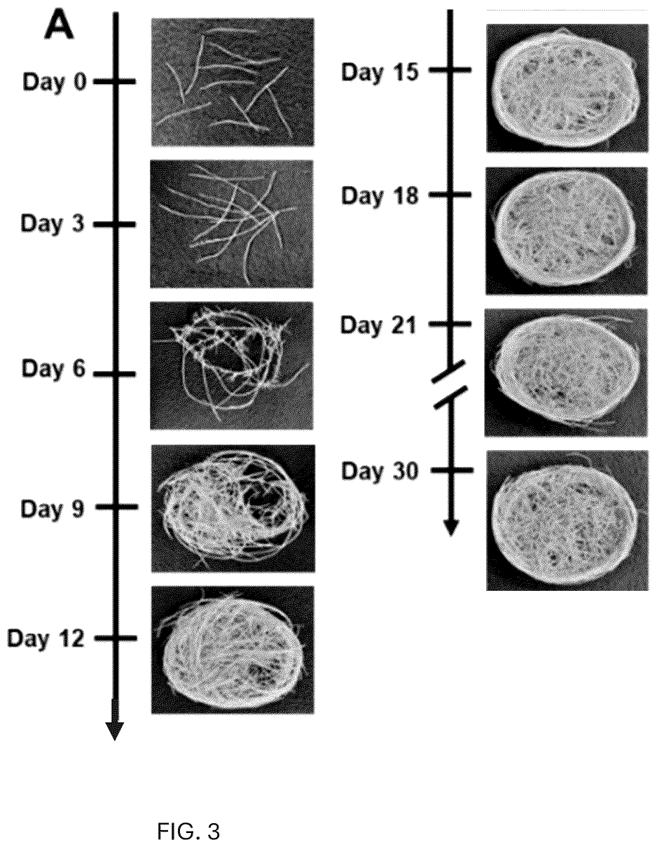

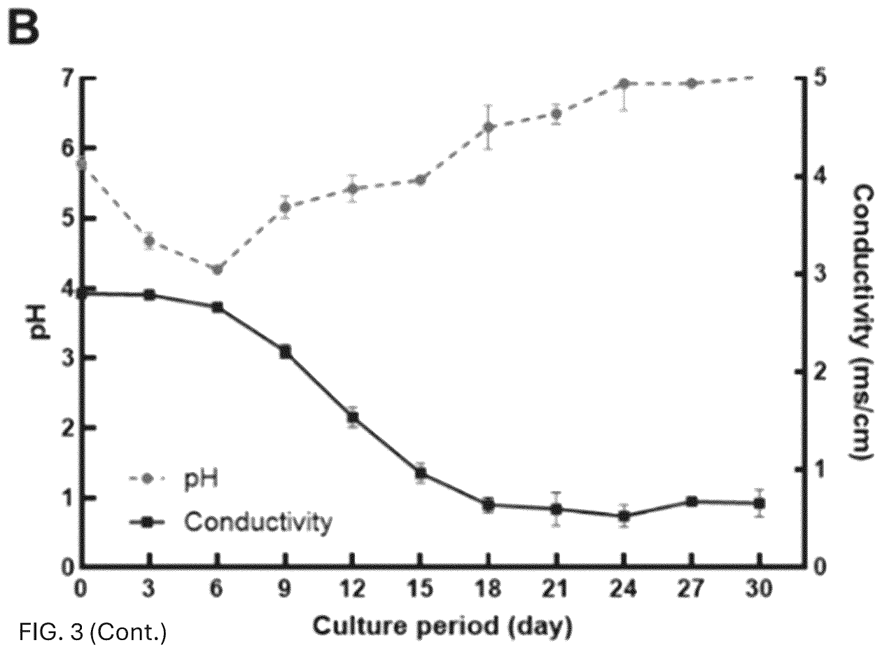

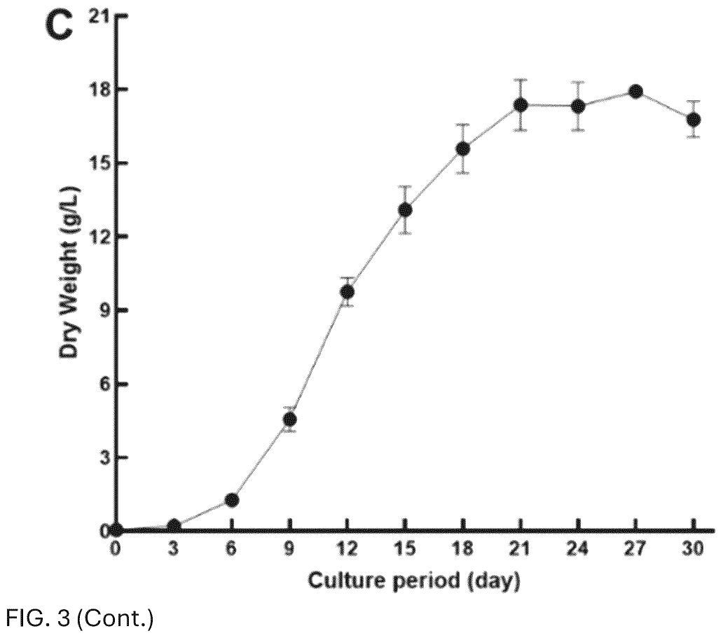

Growth Kinetics of Hairy Roots of B. orellana. To identify the growth stages of B. orellana hairy roots, a growth curve was established over 30 d in liquid culture (FIG. 3A). The growth curve was established to determine the optimal day for subculture and elicitation of the B. orellana hairy roots. During this period, pH and conductivity were measured to monitor nutrient uptake by hairy roots (FIG. 3B). The pH of the medium decreased over the first three days of culture and then gradually increased during the remaining growth period. In contrast, conductivity decreased until the roots reached their peak biomass, after which it remained constant, indicating nutrient utilization by the hairy roots throughout the 30 d. The growth curve showed a 3-day lag phase, followed by exponential growth from days 3 to 21, and a stationary phase from days 21 to 30 (FIG. 3C). The highest biomass of 17.92±0.27 g DW/L was recorded at 27 days. The doubling time, growth index, and specific growth rate of the hairy roots were calculated as 2.5 days, 303.9, and 0.27 days-1, respectively. The specific growth rate of the B. orellana hairy roots was similar to that of pigeon pea hairy roots (0.28 days-1) but lower than that of peanut hairy roots (0.31 days-1).^{25,27}

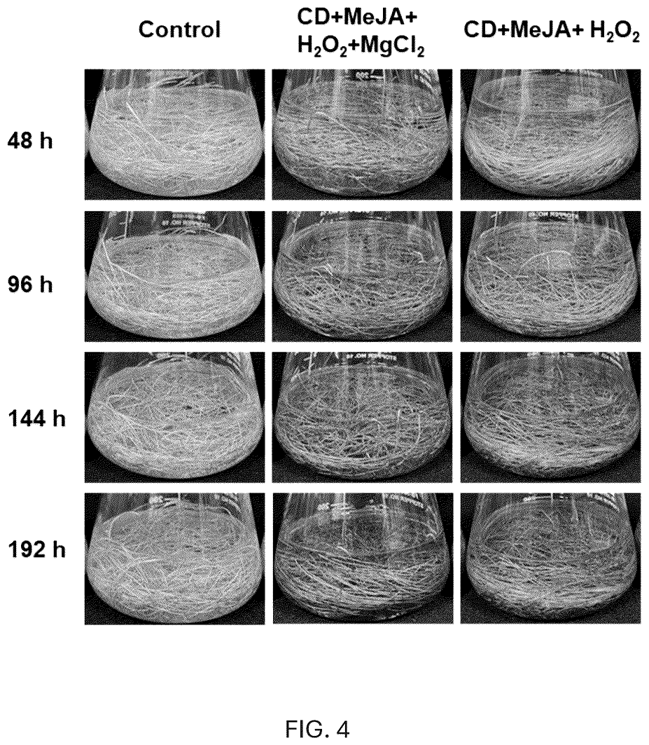

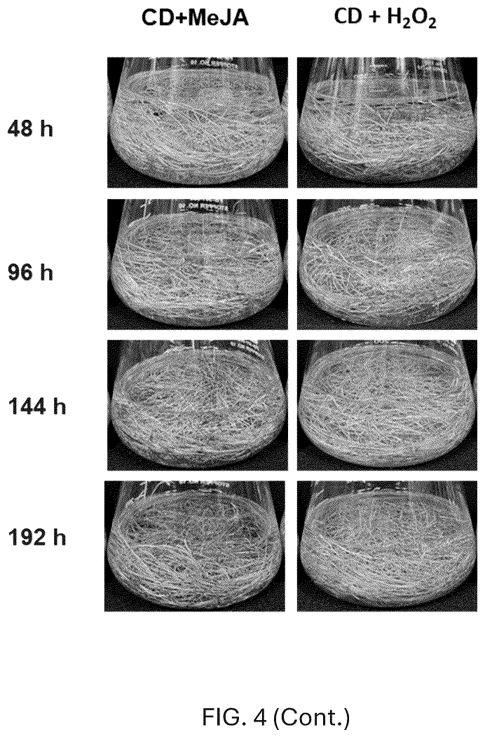

Change in the Phenotype of B. orellana Hairy Roots Upon Elicitation. In the Examples, hairy roots of B. orellana at 12 days were exposed to combined treatments of various elicitors with different time courses to investigate their involvement in the production of triterpenoids. The hairy root cultures treated with CD+MeJA+H₂O₂+MgCl₂ and CD+MeJA+H₂O₂ for 192 h showed the more intense color changes compared to the other combinations (FIG. 4). The culture medium changed from colorless to brownish in treated roots compared to that in untreated hairy root cultures. Similarly, the elicited hairy root tissues exhibited a brownish color change compared to the non-elicited controls. These observations are consistent with previous studies with peanut, pigeon pea, and mulberry hairy roots treated with combinations of CD, MeJA, H₂O₂, and MgCl₂, where the color of the culture medium shifted to yellow following elicitor treatment.²⁶⁻²⁸

Effect of Elicitor Treatment on Triterpenoid Yield in the Culture Medium. The yield of inducible triterpenoids upon treatment of B. orellana hairy root cultures with the different combinations of elicitors for varying periods was determined by HPLC (FIG. 5). Interestingly, arjunolic acid was detected in the culture medium of hairy roots treated with different combinations of elicitors, including in the control group. However, 2α,6β-dihydroxybetulinic acid, maslinic acid, and oleanolic acid were detected in the culture medium of the elicited cultures, but not in the control group, suggesting they were induced upon elicitation.

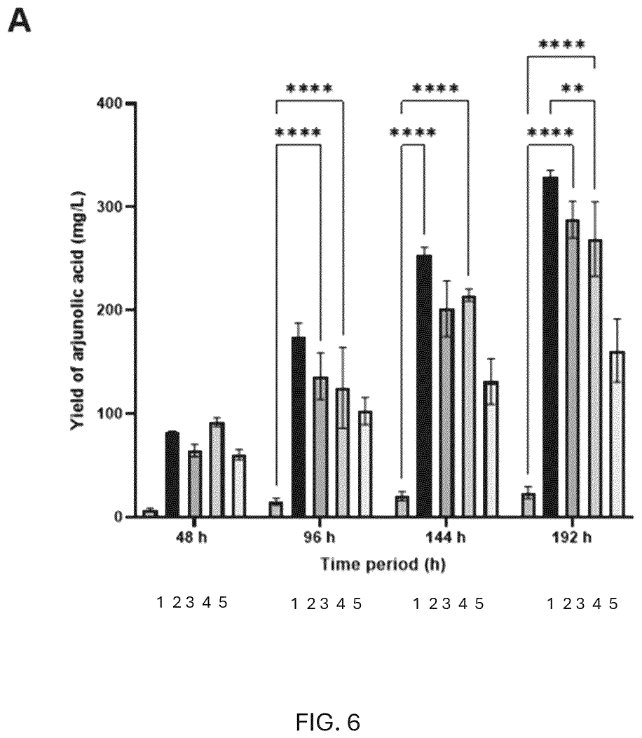

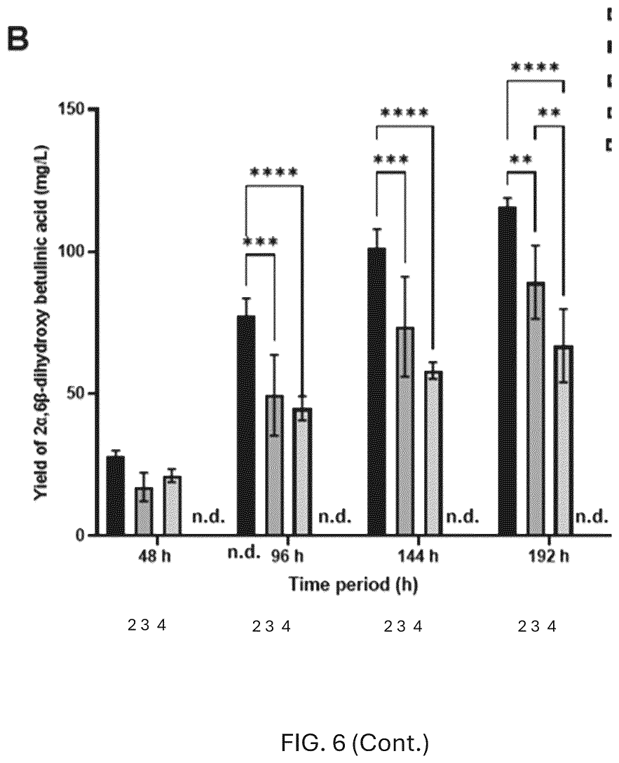

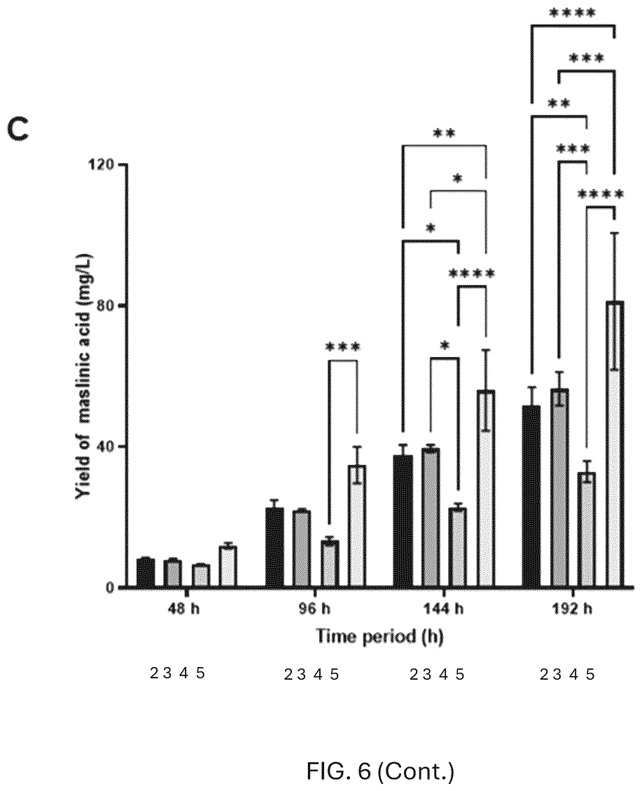

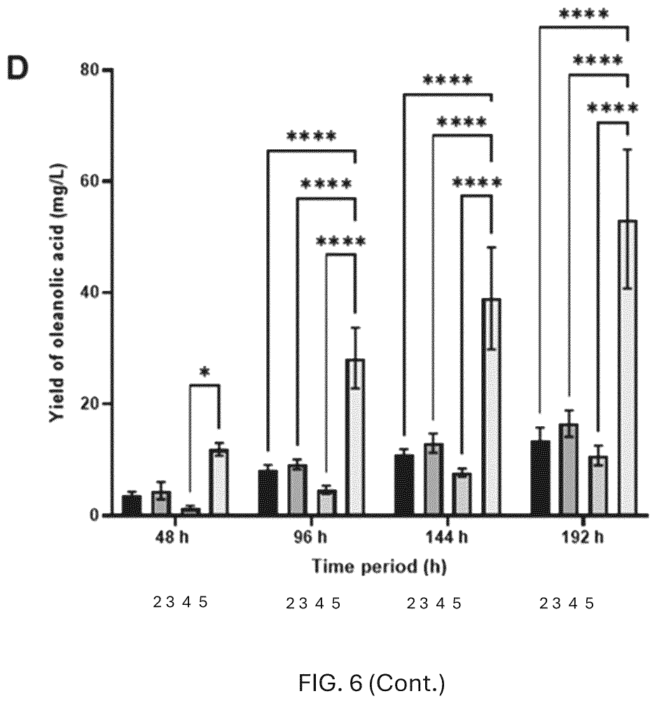

Among the triterpenoids identified, arjunolic acid was the predominant compound in the elicited culture medium (FIG. 6). Arjunolic acid exhibited a maximum yield of 329.43±6.43 mg/L in hairy roots treated with CD+MeJA+H₂O₂+MgCl₂ for 192 h. Interestingly, arjunolic acid was detected in the culture medium of the control group with the yield of 23.96±5.88 mg/L at 192 h. The yield of arjunolic acid in the elicited medium was approximately 15 times higher as compared to that in the control group. Similarly, the yield of 2α,6β-dihydroxybetulinic acid was 115.92±3.01 mg/L obtained in hairy roots treated with CD+MeJA+H₂O₂+MgCl₂ for 192 h, which expressed substantially higher levels compared to other elicitor treatment groups. However, 2α,6β-dihydroxybetulinic acid was not detected in the control group or hairy roots treated with CD+H₂O₂. The yields of maslinic acid (81.23±18.44 mg/L) and oleanolic acid (53.21±12.52 mg/L) were detected in B. orellana hairy roots treated with combination CD+H₂O₂ for 192 h. These yields were substantially higher than those of the other treatment groups. Maslinic acid and oleanolic acid were not detected in the control group. The combination of CD+MeJA+H₂O₂+MgCl₂ was effective to produce arjunolic acid and 2α,6β-dihydroxybetulinic acid, whereas the combination of CD+H₂O₂ was effective to produce maslinic acid and oleanolic acid. In summary, terpenoids were selectively enhanced when hairy roots were treated with various combinations of elicitors.

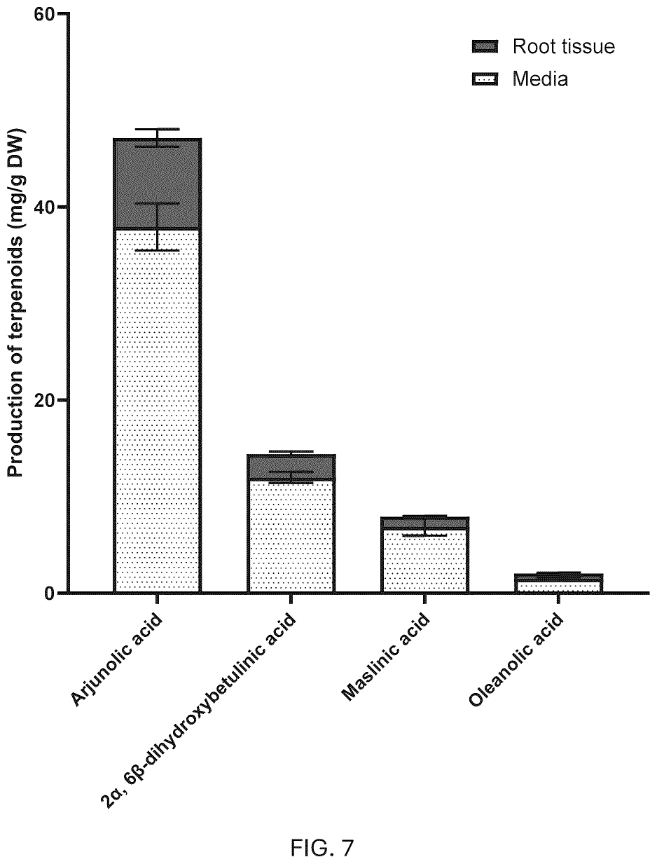

Total Yield of Triterpenoids in the Hairy Root Culture System. The total yield of triterpenoids from the B. orellana hairy root cultures was determined by treating the roots with different combinations of elicitors for 192 h. Three triterpenoids, alphitolic acid, 68-hydroxybetulinic acid, and betulinic acid, were not obtained in sufficient quantities from the culture medium extract for quantification. Arjunolic acid, 2α,6β-dihydroxybetulinic acid, maslinic acid, and oleanolic acid were quantified in the elicited culture medium of B. orellana hairy roots treated with a combination of CD, MeJA, H₂O₂, and MgCl₂. Among these four terpenoids, maslinic acid was not detected in the non-elicited control medium or tissues, whereas arjunolic acid, 2α,6β-dihydroxybetulinic acid, and maslinic acid were detected in the non-elicited hairy root tissues (FIG. 7).

Arjunolic acid exhibited a yield of 47.93±1.83 mg/g DW in the triterpenoid-rich extract of elicited B. orellana hairy root cultures treated with a combination of CD, MeJA, H₂O₂, and MgCl₂. Approximately 80% of the total arjunolic acid was secreted into the culture medium. The second-highest yield was observed for 2α,6β-dihydroxybetulinic acid, which reached 14.39±0.44 mg/g DW, 83% of which was detected in the culture medium. The total yields of maslinic acid and oleanolic acid were 7.93±0.65 mg/g DW and 2.03±0.12 mg/g DW, respectively, with approximately 87% of maslinic acid and 74% of oleanolic acid secreted into the culture medium. The overall triterpenoid yield in the elicited B. orellana hairy root cultures treated with the CD+MeJA+H₂O₂+MgCl₂ combination was approximately 71.5 mg/g DW.

Arjunolic acid, 2α,6β-dihydroxybetulinic acid, and oleanolic acid were also detected in tissues of non-elicited hairy root cultures. The yields of arjunolic acid, 2α,6β-dihydroxybetulinic acid, and oleanolic acid in the control root tissue were 4.76±0.44 mg/g DW, 0.13±0.03 mg/g DW, and 1.17±0.07 mg/g DW, respectively. The total yield of arjunolic acid in the non-elicited control hairy roots was 5.70±0.31 mg/g DW. Compared to the non-elicited controls, the total yields of arjunolic acid, 2α,6β-dihydroxybetulinic acid, and oleanolic acid were approximately 8-, 103-, and 1.7 times higher, respectively. The triterpenoid-rich extract from elicited B. orellana hairy root cultures contained 35.08% arjunolic acid, 10.2% 2α,6β-dihydroxybetulinic acid, 7.48% maslinic acid, 1.92% oleanolic acid, and other triterpenoids. This finding suggests that elicitation substantially enhances terpenoid production in the hairy roots of B. orellana. This represents the reported yield for arjunolic acid, 2α,6β-dihydroxybetulinic acid, and maslinic acid.

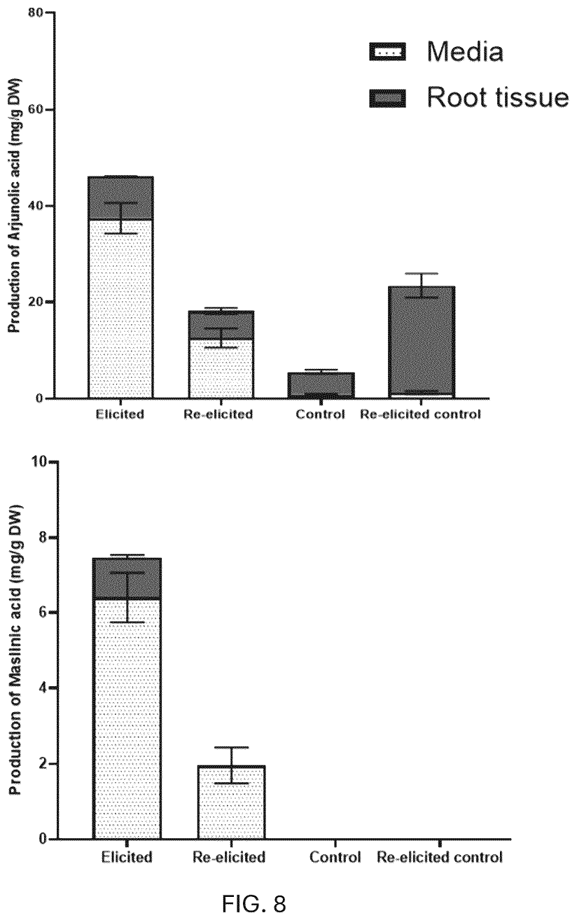

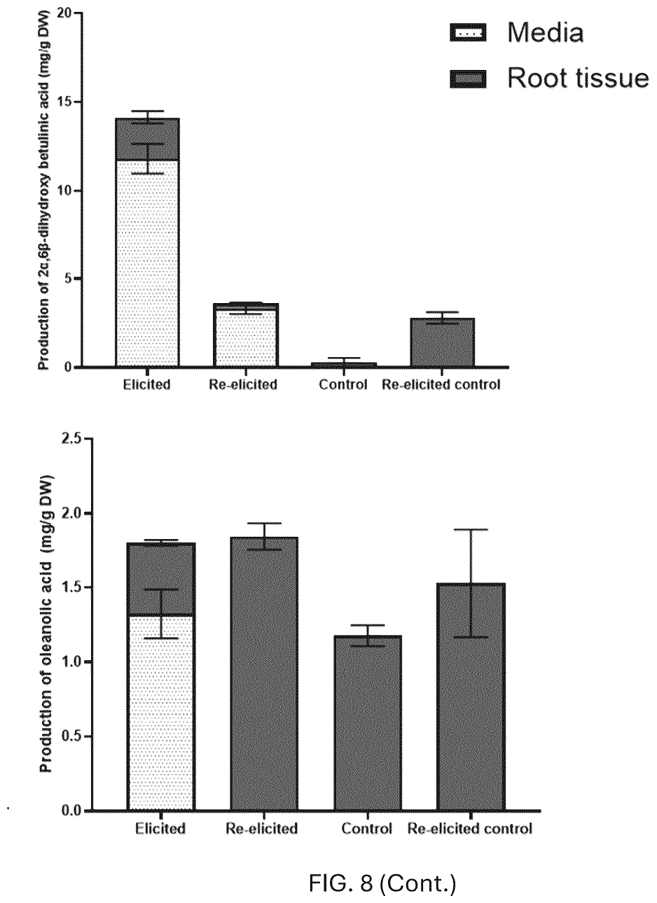

Re-elicitation of Elicited B. orellana Hairy Root Cultures. The elicited hairy root cultures of B. orellana were treated with a combination of CD+MeJA+H₂O₂+MgCl₂ for an additional 192 h to determine the potential of the hairy root system to respond to the second elicitor treatment. The re-elicited cultures had arjunolic acid (18.13±1.32 mg/g DW), 2α,6β-dihydroxybetulinic acid (3.61±0.18 mg/g DW), maslinic acid (1.96±0.23 mg/g DW), and oleanolic acid (1.84±0.04 mg/g DW). Interestingly, these combined yields from re-elicited culture medium and root tissue were lower than those obtained after the first elicitation (FIG. 8). The yields of arjunolic acid, 2a, 68-dihydroxybetulinic acid, maslinic acid, and oleanolic acid increased by 28, 20, 20, and 48%, respectively, upon re-elicitation. The total yield of triterpenoids in the re-elicited B. orellana hairy root was 25.6 mg/g DW, which was approximately 36% in addition to the first elicitation. Interestingly, the accumulation of arjunolic acid (22.25±2.58 mg/g DW), 2α,6β-dihydroxybetulinic acid (2.79±0.32 mg/g DW), and oleanolic acid (1.53±0.36 mg/g DW) increased in the non-treated hairy roots grown for 384 h when compared to the non-treated hairy roots grown for 192 h. Previously, re-elicitation of peanut and pigeon pea hairy roots resulted in substantial accumulation of stilbene and isoflavones, respectively.^{25,29} The increment in yield of isoflavonoids after re-elicitation of pigeon pea hairy root system was 46%, which was higher as compared to re-elicitation of B. orellana hairy roots.

Purification of Triterpenoids and Semi-synthesis of Acetylated Triterpenoid Derivatives. To enhance the production and secretion of terpenoids in the culture medium of the B. orellana hairy root cultures, hairy roots were co-treated with a combination of MeJA, CD, H₂O₂, and MgCl₂ for 192 h. A triterpenoid-rich extract was obtained by extracting the culture medium with ethyl acetate. Then, the extract was fractionated using normal- and reversed-phase column chromatography, and purification was achieved using reversed-phase semi-preparative HPLC on a C-18 column. The triterpenoids identified included arjunolic acid, 2α,6β-dihydroxybetulinic acid, alphitolic acid, maslinic acid, 6β-hydroxybetulinic acid, betulinic acid, and oleanolic acid. The structures of the terpenoids were elucidated by comparing the obtained MS and NMR spectra with previous reports (Figures S3-S23).³⁰⁻³⁵ In the Examples, 2α,6β-dihydroxybetulinic acid and 6β-hydroxybetulinic acid were reported for the first time in the genus Bixa. The triterpenoid 2α,6β-dihydroxybetulinic acid was reported for the first time from the seeds of Combretum quadrangulare, and 6β-hydroxybetulinic acid was reported for the first time from the leaves of Licanina pyrifolia.^{31,32} Additionally, an acetylation reaction was conducted to produce the acetylated derivatives of arjunolic acid and maslinic acid, both obtained with high yields, and their structure was confirmed using NMR and MS spectroscopy (Figure S24-S29). The acetylated derivatives were semi-synthesized to increase their lipophilicity, passive membrane permeability, and enhance their bioavailability.³⁶

Arjunolic acid has been reported previously from the stem bark of Terminalia arjuna.³⁷ Oleanolic acid and maslinic acid have been isolated from pomace olive, whereas alphitolic acid and betulinc acid were reported from Zyziphi fructus.^{38,39} Both arjunolic and maslinic acids have been previously reported from hairy roots of B. orellana elicited with MeJA.¹² Arjunolic acid, maslinic acid, betulinic acid, and oleanolic acid were reported to have antibacterial, anticancer, anti-inflammatory, cardioprotection, antidiabetic, and other properties.^{16,40-42} Similarly, alphitolic acid has been reported to demonstrate anti-inflammatory, anticancer, and antiparasitic activities.⁴³ Additionally, 2α,6β-dihydroxybetulinic acid, and 6β-hydroxybetulinic acid have demonstrated hepatoprotective, antiplatelet, and vasorelaxant effects.⁴⁴⁻⁴⁶

Anti-inflammatory Activity of B. orellana Triterpenoids. RAW 264.7 murine macrophages were stimulated with LPS to evaluate the anti-inflammatory effects of the triterpenoids detected in the elicited B. orellana hairy root cultures. Additionally, the anti-inflammatory activities of the semi-synthesized derivatives of arjunolic acid and maslinic acid were assessed. Before evaluating their anti-inflammatory properties, the cytotoxic effects of the terpenoids on RAW 264.7 macrophages were determined (FIG. 9). The IC₅₀ values for the triterpenoids ranged from 25 to 50 μM, indicating that all compounds were non-toxic, even at higher concentrations. Therefore, the anti-inflammatory activities of these compounds were tested at concentrations of 20 μM and 35 μM except for the acetylated derivative of arjunolic acid.

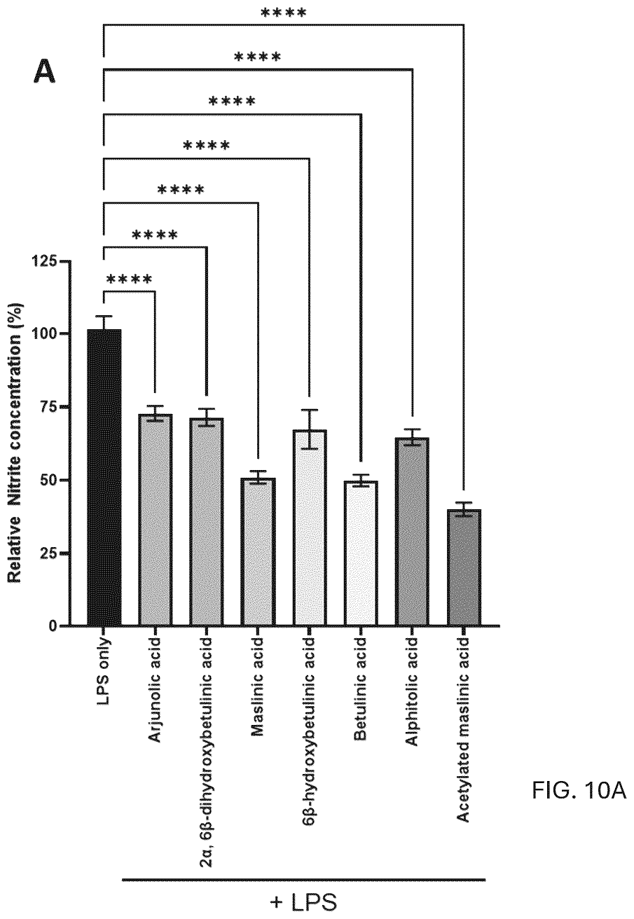

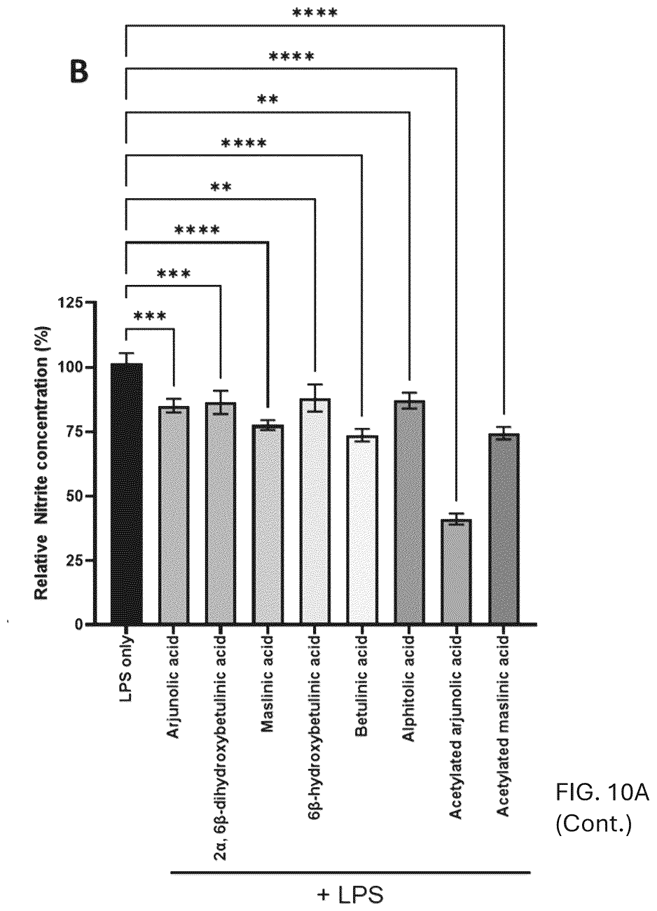

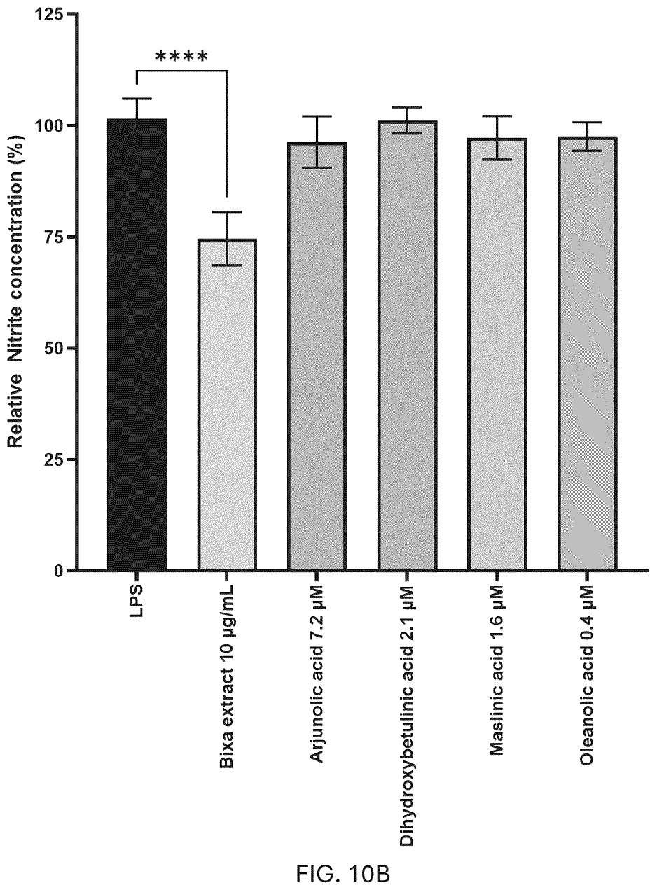

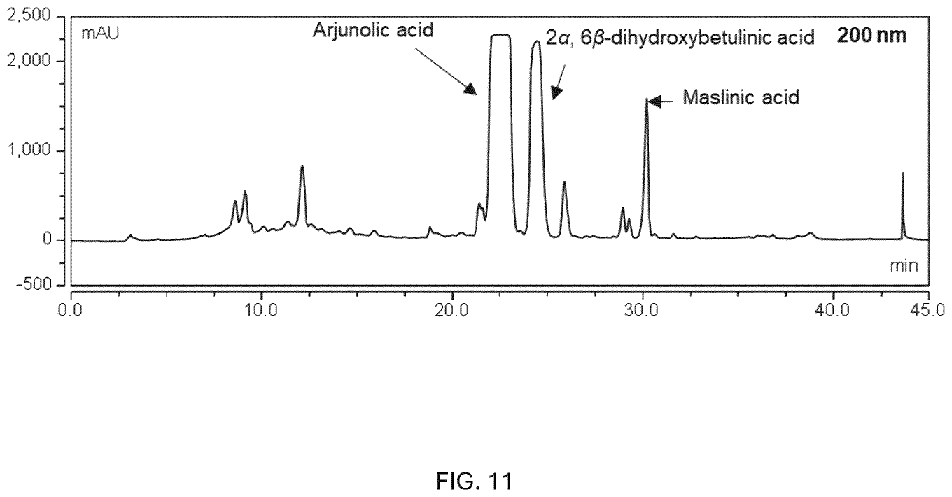

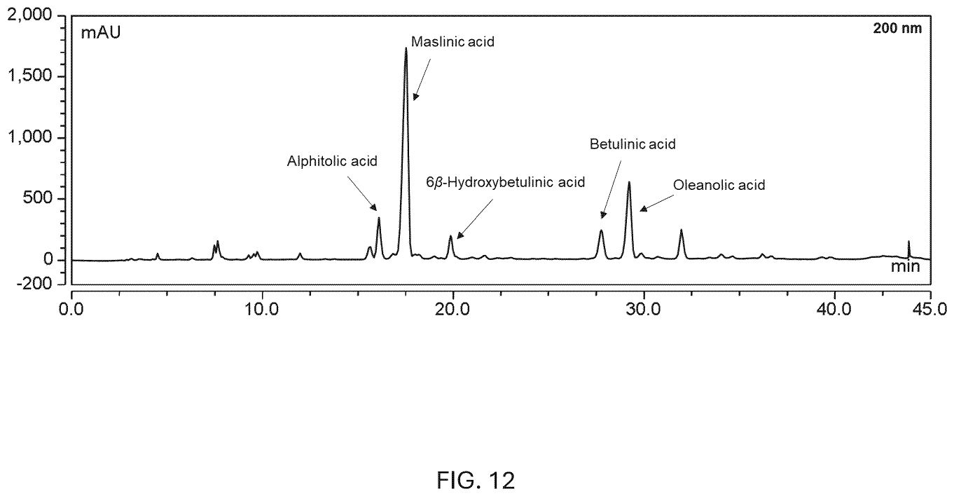

The anti-inflammatory activity of triterpenoids was assessed by measuring their effect on nitrite levels using the Griess reagent system after LPS treatment. All triterpenoids reduced nitrite levels at concentrations of 35 and 20 μM compared to cells treated with LPS alone (FIG. 10). The semi-synthetic acetylated derivative of arjunolic acid exhibited the substantial reduction in nitrite levels at both concentrations compared with the other triterpenoids. In cells pre-treated with 20 μM of the acetylated derivative of arjunolic acid, nitrite levels were reduced to 20.54±1.76%, following LPS-only treatment. Similarly, the acetylated derivative of maslinic acid showed a greater reduction in the level of nitrite at both 35 μM (40.09±2.32%) and 20 μM (74.48±2.44%) relative to LPS-only. At 35 μM concentration, the acetylated derivative of maslinic acid showed a substantial reduction in the nitrite level compared to maslinic acid (51.03±2.12%). Similarly, the acetylated derivative of arjunolic acid showed substantially higher reduction in the nitrite levels as compared to that of arjunolic acid (72.83±2.53%). The acetylated derivatives of both arjunolic acid and maslinic acid demonstrated higher activities than their parent molecules. This suggests that adding acetyl groups to arjunolic acid and maslinic acid increases their anti-inflammatory properties.

Among the triterpenoids isolated from the elicited B. orellana hairy root culture medium, maslinic acid (51.03±2.12%) and betulinic acid (49.93±1.99%) showed the greatest nitrite reduction at a concentration of 35 μM following LPS-only treatment. Similarly, at 20 μM, betulinic acid demonstrated the greatest reduction in nitrite levels (73.71±2.45%) compared to the LPS-only treatment. The anti-inflammatory activity for maslinic acid and betulinic acid against LPS-stimulated RAW 264.7 macrophages has been reported previously as well.^{47,48}

In terms of structure-activity relationships, there was no substantial difference in the anti-inflammatory activity between oleanane-type and lupane-type terpenoids. Oleanane-type terpenoids include arjunolic acid, maslinic acid, and betulinic acid, whereas lupane-type terpenoids include 2α,6β-dihydroxybetulinic acid, 6β-hydroxybetulinic acid, alphitolic acid, and betulinic acid. Interestingly, the number of hydroxyl groups in the terpene backbone also influences anti-inflammatory activity. For example, maslinic acid exhibited higher anti-inflammatory activity than arjunolic acid, and a similar trend was observed for alphitolic acid and betulinic acid. Furthermore, the addition of an acetyl group to arjunolic acid and maslinic acid substantially enhanced their anti-inflammatory activities. Previously, acetylation of betulinic acid has resulted in an increment of their anti-inflammatory activity by reducing the nitrite level.⁴⁹

Antimicrobial Activity of B. orellana Triterpenoids. The antimicrobial activity of terpenoids from elicited B. orellana hairy roots was tested against two Gram-negative bacteria and several Gram-positive bacteria. Maslinic acid demonstrated the greatest antimicrobial activity against several Gram-positive bacteria. The MIC of maslinic acid ranged between 6.25 μg/mL and 12.5 μg/mL (Table 1). Maslinic acid demonstrated potent antimicrobial activity with MIC value of 6.25 μg/mL against Enterococcus faecalis ATCC 29212, E. faecalis ATCC 51299 (VRE), E. faecium ATCC 700221 (VRE), Staphylococcus aureus ATCC 25923, and Streptococcus mutans UA159. It also exhibited antibacterial activity against Bacillus subtilis ATCC 6623, S. aureus ATCC 35591 (MRSA), and S. aureus ATCC00699 (MRSA), with MIC value of 12.5 μg/mL. Similar results were reported for maslinic acid against S. aureus, MRSA, E. faecalis, and S. mutans.⁵⁰

Interestingly, oleanolic acid demonstrated substantial antimicrobial activity against vancomycin-resistant E. faecium (VRE) and E. faecalis, with a MIC value of 6.25 μg/mL. Then, 6β-hydroxybetulinic acid demonstrated considerable antimicrobial activity against S. mutans and B. subtilis with MIC values of 12.5 μg/mL and 25 μg/mL respectively. This is the first report on the antimicrobial activity of 6β-hydroxybetulinic acid against B. subtilis and S. mutans. Other triterpenoids demonstrated weak antimicrobial activity, with MIC values >50 μg/mL. Additionally, the semi-synthetic derivatives of arjunolic acid and maslinic acid did not exhibit any notable antimicrobial activity against Gram-positive and Gram-negative bacteria. This suggests that adding an acetyl group to arjunolic acid and maslinic acid decreases their antimicrobial activity. In addition, lupane-type terpenoids demonstrated lower antimicrobial activity than oleanane-type terpenoids isolated from B. orellana hairy roots.

TABLE 1
Antimicrobial activity of triterpenoids from Bixa orellana hairy root cultures

| | MIC (μg/mL) | | | |
|---|---|---|---|---|
| | | 2α,6β- | | |
| | Arjunolic | dihydroxybetulinic | Maslinic | Oleanolic |
| Microorganisms | acid | acid | acid | acid |
| Bacillus subtilis | 50 | >100 | 12.5 | >100 |
| ATCC 6623^{a} | | | | |
| Escherichia coli | >100 | >100 | >100 | >100 |
| ATCC 700603^{b} | | | | |
| Enterococcus | 50 | >100 | 6.25 | 25 |
| faecalis | | | | |
| ATCC 29212^{a} | | | | |
| Enterococcus | 50 | >100 | 6.25 | 6.25 |
| faecalis | | | | |
| ATCC51299 | | | | |
| (VRE)^{b} | | | | |
| Enterococcus | 50 | >100 | 6.25 | 6.25 |
| faecium | | | | |
| ATCC700221 | | | | |
| (VRE)^{b} | | | | |
| Klebsiella | >100 | >100 | >100 | >100 |
| pneumoniae | | | | |
| ATCC 700603^{b} | | | | |
| Staphylococcus | 50 | >100 | 6.25 | >100 |
| aureus | | | | |
| ATCC 25923^{a} | | | | |
| Staphylococcus | 100 | >100 | 12.5 | >100 |
| aureus | | | | |
| ATCC 35591 | | | | |
| (MRSA)^{a} | | | | |
| Staphylococcus | >100 | >100 | 12.5 | >100 |
| aureus | | | | |
| ATCC00699 | | | | |
| (MRSA)^{b} | | | | |
| Streptococcus | 25 | 100 | 6.25 | >100 |
| mutans | | | | |
| UA159^{a} | | | | |

| | | 6β- | | |
|---|---|---|---|---|
| | Betulinic | hydroxybetulinic | Alphitolic | |
| | acid | acid | acid | Reference |
| Bacillus subtilis | >100 | 25 | >100 | 0.31^{c} |
| ATCC 6623^{a} | | | | |
| Escherichia coli | >100 | >100 | >100 | 2.5^{d} |
| ATCC 700603^{b} | | | | |
| Enterococcus | >100 | >100 | >100 | 5^{c} |
| faecalis | | | | |
| ATCC 29212^{a} | | | | |
| Enterococcus | >100 | 100 | >100 | 5^{e} |
| faecalis | | | | |
| ATCC51299 | | | | |
| (VRE)^{b} | | | | |
| Enterococcus | >100 | >100 | >100 | 5^{e} |
| faecium | | | | |
| ATCC700221 | | | | |
| (VRE)^{b} | | | | |
| Klebsiella | >100 | >100 | >100 | 2.5^{d} |
| pneumoniae | | | | |
| ATCC 700603^{b} | | | | |
| Staphylococcus | >100 | >100 | >100 | 0.62^{c} |
| aureus | | | | |
| ATCC 25923^{a} | | | | |
| Staphylococcus | >100 | >100 | >100 | 2.5^{c} |
| aureus | | | | |
| ATCC 35591 | | | | |
| (MRSA)^{a} | | | | |
| Staphylococcus | >100 | >100 | >100 | 5^{c} |
| aureus | | | | |
| ATCC00699 | | | | |
| (MRSA)^{b} | | | | |
| Streptococcus | >100 | 12.5 | >100 | 1.25^{c} |
| mutans | | | | |
| UA159^{a} | | | | |

^{a}Gram-positive bacteria.
^{b}Gram-negative bacteria.
^{c}Vancomycin hydrochloride.
^{d}Colistin sulfate.
^{e}Daptomycin

Cytotoxicity Activity of B. orellana Triterpenoids. The cytotoxic effects of triterpenoids from elicited B. orellana hairy root cultures were evaluated against MCF-7 breast cancer cells (FIG. 9B). MCF-7 cells were treated with triterpenoids at concentrations ranging from 3.12 to 100 μM for 72 h. The cytotoxic effects were detected using the RealTime-Glo MT Cell Viability Assay reagent, with cells treated with 0.05% DMSO serving as controls. Acetylation of arjunolic acid increased the cytotoxicity to MCF-7 breast cancer cells compared to the parent compound.

Summary. In conclusion, seven distinct terpenoids were identified in the culture medium of Bixa hairy roots. Among them, two terpenoids, 2α,6β-dihydroxybetulinic acid and 68-hydroxybetulinic acid, were reported for the first time in B. orellana. Additionally, previously known triterpenoids, including arjunolic acid, alphitolic acid, maslinic acid, betulinic acid, and oleanolic acid, have also been identified. The structures of these terpenoids were elucidated by MS and NMR analyses. Elicitation and re-elicitation strategies substantially enhanced the yield of triterpenoids in the hairy roots of this species. The triterpenoid-rich extract consisted of more than 50% triterpenoids. Furthermore, a semi-synthetic approach was employed to obtain acetylated derivatives of arjunolic acid and maslinic acid. All identified triterpenoids demonstrated anti-inflammatory activity, with maslinic acid showing notable antimicrobial activity against vancomycin-resistant Enterococcus faecalis and E. faecium, as well as methicillin-resistant Staphylococcus aureus. These findings underscore the value of B. orellana hairy root cultures as a sustainable platform for the high-yield production of bioactive triterpenoids.

### REFERENCES

(1) Scotter, M. The Chemistry and Analysis of Annatto Food Colouring: A Review. Food Additives & Contaminants: Part A 2009, 26 (8), 1123-1145. doi.org/10.1080/02652030902942873.
(2) Shahid-ul-Islam; Rather, L. J.; Mohammad, F. Phytochemistry, Biological Activities and Potential of Annatto in Natural Colorant Production for Industrial Applications-A Review. J Adv Res 2016, 7 (3), 499-514. doi.org/10.1016/j.jare.2015.11.002.
(3) Saha, P. Das; Sinha, K. Natural Dye from Bixa Seeds as a Potential Alternative to Synthetic Dyes for Use in Textile Industry. Desalination Water Treat 2012, 40 (1), 298-301. doi.org/10.5004/dwt.2012.3086.
(4) Tupuna, D. S.; Paese, K.; Guterres, S. S.; Jablonski, A.; Flôres, S. H.; Rios, A. de O. Encapsulation Efficiency and Thermal Stability of Norbixin Microencapsulated by Spray-Drying Using Different Combinations of Wall Materials. Ind Crops Prod 2018, 111, 846-855. doi.org/10.1016/j.indcrop.2017.12.001.
(5) Rao, P. P.; Satyanarayana, A.; Balaswamy, K.; Jyothirmayi, T.; Nagender, A.; Rao, D. G. Application of Annatto Dye Formulation in Bakery and Extruded Food Products. Journal of Foodservice 2007, 18 (2), 53-58. doi.org/10.1111/j.1745-4506.2007.00046.x.
(6) Sharma, P.; Segat, A.; Kelly, A. L.; Sheehan, J. J. Colorants in Cheese Manufacture: Production, Chemistry, Interactions, and Regulation. Compr Rev Food Sci Food Saf 2020, 19 (4), 1220-1242. doi.org/10.1111/1541-4337.12519.
(7) Lima Viana, J.; Zagmignan, A.; Lima Lobato, L. F.; Gomes Abreu Jr., A.; da Silva, L. C. N.; Sá, J. C. de; Monteiro, C. de A.; Lago, J. H. G.; Gonçalves, L. M.; Carvalho, R. C.; Neto, L. G. L.; de Sousa, E. M. Hydroalcoholic Extract and Ethyl Acetate Fraction of Bixa Orellana Leaves Decrease the Inflammatory Response to Mycobacterium abscessus Subsp. massiliense. Evidence-Based Complementary and Alternative Medicine 2018, 2018 (1), 6091934. doi.org/10.1155/2018/6091934.
(8) Vilar, D. de A.; Vilar, M. S. de A.; Moura, T. F. A. de L. e; Raffin, F. N.; Oliveira, M. R. de; Franco, C. F. de O.; de Athayde-Filho, P. F.; Diniz, M. de F. F. M.; Barbosa-Filho, J. M. Traditional Uses, Chemical Constituents, and Biological Activities of Bixa orellana L.: A Review. The Scientific World Journal 2014, 2014 (1), 857292. doi.org/10.1155/2014/857292.
(9) Shilpi, J. A.; Taufiq-Ur-Rahman, Md.; Uddin, S. J.; Alam, Md. S.; Sadhu, S. K.; Seidel, V. Preliminary Pharmacological Screening of Bixa orellana L. Leaves. J Ethnopharmacol 2006, 108 (2), 264-271. doi.org/10.1016/j.jep.2006.05.008.
(10) Coelho dos Santos, D.; Silva Barboza, A. da; Ribeiro, J. S.; Rodrigues Junior, S. A.; Campos, Â. D.; Lund, R. G. Bixa orellana L. (Achiote, Annatto) as an Antimicrobial Agent: A Scoping Review of Its Efficiency and Technological Prospecting. J Ethnopharmacol 2022, 287, 114961. doi.org/10.1016/j.jep.2021.114961.
(11) Stohs, S. J. Safety and Efficacy of Bixa orellana (Achiote, Annatto) Leaf Extracts. Phytotherapy Research 2014, 28 (7), 956-960. doi.org/10.1002/ptr.5088.
(12) Zhai, B.; Clark, J.; Ling, T.; Connelly, M.; Medina-Bolivar, F.; Rivas, F. Antimalarial Evaluation of the Chemical Constituents of Hairy Root Culture of Bixa orellana L. Molecules 2014, 19 (1), 756-766. doi.org/10.3390/molecules19010756.
(13) Bakrim, S.; Benkhaira, N.; Bourais, I.; Benali, T.; Lee, L.-H.; El Omari, N.; Sheikh, R. A.; Goh, K. W.; Ming, L. C.; Bouyahya, A. Health Benefits and Pharmacological Properties of Stigmasterol. Antioxidants 2022, 11 (10). doi.org/10.3390/antiox11101912.
(14) Ambavade, S. D.; Misar, A. V; Ambavade, P. D. Pharmacological, Nutritional, and Analytical Aspects of β-Sitosterol: A Review. Orient Pharm Exp Med 2014, 14 (3), 193-211. doi.org/10.1007/s13596-014-0151-9.
(15) Yan, R.; Liu, L.; Huang, X.; Quan, Z.-S.; Shen, Q.-K.; Guo, H.-Y. Bioactivities and Structure-Activity Relationships of Maslinic Acid Derivatives: A Review. Chem Biodivers 2024, 21 (2), e202301327. doi.org/10.1002/cbdv.202301327.
(16) Ghosh, J.; Sil, P. C. Arjunolic Acid: A New Multifunctional Therapeutic Promise of Alternative Medicine. Biochimie 2013, 95 (6), 1098-1109. doi.org/10.1016/j.biochi.2013.01.016.
(17) Shahid-ul-Islam; Rather, L. J.; Mohammad, F. Phytochemistry, Biological Activities and Potential of Annatto in Natural Colorant Production for Industrial Applications-A Review. J Adv Res 2016, 7 (3), 499-514. doi.org/10.1016/j.jare.2015.11.002.
(18) Zhu, X.; Liu, X.; Liu, T.; Wang, Y.; Ahmed, N.; Li, Z.; Jiang, H. Synthetic Biology of Plant Natural Products: From Pathway Elucidation to Engineered Biosynthesis in Plant Cells. Plant Commun 2021, 2 (5), 100229. doi.org/10.1016/j.xplc.2021.100229.
(19) Vanhakylä, S.; Salminen, J.-P. Seasonal Variation in Plant Polyphenols and Related Bioactivities across Three Years in Ten Tree Species as Visualized by Mass Spectrometric Fingerprint Mapping. Molecules 2023, 28 (16). doi.org/10.3390/molecules28166093.
(20) Hussain, M. J.; Abbas, Y.; Nazli, N.; Fatima, S.; Drouet, S.; Hano, C.; Abbasi, B. H. Root Cultures, a Boon for the Production of Valuable Compounds: A Comparative Review. Plants 2022, 11 (3). doi.org/10.3390/plants11030439.
(21) Rogowska, A.; Szakiel, A. Enhancement of Phytosterol and Triterpenoid Production in Plant Hairy Root Cultures-Simultaneous Stimulation or Competition? Plants 2021, 10 (10). doi.org/10.3390/plants10102028.
(22) Marsh, Z.; Yang, T.; Nopo-Olazabal, L.; Wu, S.; Ingle, T.; Joshee, N.; Medina-Bolivar, F. Effect of Light, Methyl Jasmonate and Cyclodextrin on Production of Phenolic Compounds in Hairy Root Cultures of Scutellaria lateriflora. Phytochemistry 2014, 107, 50-60. doi.org/10.1016/j.phytochem.2014.08.020.
(23) Condori, J.; Sivakumar, G.; Hubstenberger, J.; Dolan, M. C.; Sobolev, V. S.; Medina-Bolivar, F. Induced Biosynthesis of Resveratrol and the Prenylated Stilbenoids Arachidin-1 and Arachidin-3 in Hairy Root Cultures of Peanut: Effects of Culture Medium and Growth Stage. Plant Physiology and Biochemistry 2010, 48 (5), 310-318. doi.org/10.1016/j.plaphy.2010.01.008.
(24) Ferrand Malatesta, M. Analyses of Constitutive and Inducible Specialized Metabolites in Tissues and Hairy Root Cultures of Annatto (Bixa orellana L.). Master Thesis, Arkansas State University, Jonesboro, 2008.
(25) Sharma, A. R.; Gajurel, G.; Ahmed, I.; Roedel, K.; Medina-Bolivar, F. Induction of the Prenylated Stilbenoids Arachidin-1 and Arachidin-3 and Their Semi-Preparative Separation and Purification from Hairy Root Cultures of Peanut (Arachis hypogaea L.). Molecules 2022, 27 (18). doi.org/10.3390/molecules27186118.
(26) Fang, L.; Yang, T.; Medina-Bolivar, F. Production of Prenylated Stilbenoids in Hairy Root Cultures of Peanut (Arachis hypogaea) and Its Wild Relatives A. Ipaensis and A. duranensis via an Optimized Elicitation Procedure. Molecules 2020, 25 (3). doi.org/10.3390/molecules25030509.
(27) Gajurel, G.; Nopo-Olazabal, L.; Hendrix, E.; Medina-Bolivar, F. Production and Secretion of Isowighteone in Hairy Root Cultures of Pigeon Pea (Cajanus cajan) Co-Treated with Multiple Elicitors. Plants 2022, 11 (6). doi.org/10.3390/plants11060834.
(28) Fang, L.; Sharma, A. R.; Aniemena, C.; Roedel, K.; Henry, F.; Moussou, P.; Samuga, A.; Medina-Bolivar, F. Elicitation of Stilbenes and Benzofuran Derivatives in Hairy Root Cultures of White Mulberry (Morus alba). Plants 2023, 12 (1). doi.org/10.3390/plants12010175.
(29) Gajurel, G.; Sharma, A. R.; Abdel-Karim, S.; Balmaceda, C.; Zulfiqar, F.; Pandey, P.; Ali, Z.; Shields, R. C.; Alam, M. A.; Medina-Bolivar, F. Enhanced Production of Anti-Inflammatory and Antibacterial Prenylated Isoflavonoids in Pigeon Pea (Cajanus cajan) Hairy Root Cultures. J Agric Food Chem 2025. doi.org/10.1021/acs.jafc.4c12406.
(30) Ramesh, A. S.; Christopher, J. G.; Radhika, R.; Setty, C. R.; Thankamani, V. Isolation, Characterisation and Cytotoxicity Study of Arjunolic Acid from Terminalia arjuna. Nat Prod Res 2012, 26 (16), 1549-1552. doi.org/10.1080/14786419.2011.566870.
(31) Adnyana, I. K.; Tezuka, Y.; Banskota, A. H.; Tran, K. Q.; Kadota, S. Three New Triterpenes from the Seeds of Combretum quadrangulare and Their Hepatoprotective Activity. J Nat Prod 2001, 64 (3), 360-363. doi.org/10.1021/np000486x.
(32) Bilia, A. R.; Morelli, I.; Mendez, J. New Lupane Derivatives from the Leaves of Licania pyrifolia. J Nat Prod 1996, 59 (3), 297-300. doi.org/10.1021/np960134j.
(33) Martinez, A.; Rivas, F.; Perojil, A.; Parra, A.; Garcia-Granados, A.; Fernandez-Vivas, A. Biotransformation of Oleanolic and Maslinic Acids by Rhizomucor miehei. Phytochemistry 2013, 94, 229-237. doi.org/10.1016/j.phytochem.2013.05.011.
(34) Park, S.; Cho, J.; Jeon, H.; Sung, S. H.; Lee, S.; Kim, S. Expedient Synthesis of Alphitolic Acid and Its Naturally Occurring 2-O-Ester Derivatives. J Nat Prod 2019, 82 (4), 895-902. doi.org/10.1021/acs.jnatprod.8b00986.
(35) SHOLICHIN, M.; YAMASAKI, K.; KASAI, R.; TANAKA, O. \hat{ }&lt;13&gt;C Nuclear Magnetic Resonance of Lupane-Type Triterpenes, Lupeol, Betulin and Betulinic Acid. Chem Pharm Bull (Tokyo) 1980, 28 (3), 1006-1008. doi.org/10.1248/cpb.28.1006.
(36) Rautio, J.; Kumpulainen, H.; Heimbach, T.; Oliyai, R.; Oh, D.; Järvinen, T.; Savolainen, J. Prodrugs: Design and Clinical Applications. Nat Rev Drug Discov 2008, 7 (3), 255-270. doi.org/10.1038/nrd2468.
(37) Verma, S. C.; Jain, C. L.; Padhi, M. M.; Devalla, R. B. Microwave Extraction and Rapid Isolation of Arjunic Acid from Erminalia arjuna (Roxb. Ex DC.) Stem Bark and Quantification of Arjunic Acid and Arjunolic Acid Using HPLC-PDA Technique. J Sep Sci 2012, 35 (13), 1627-1633. doi.org/10.1002/jssc.201200083.
(38) Juan, M. E.; Planas, J. M.; Ruiz-Gutierrez, V.; Daniel, H.; Wenzel, U. Antiproliferative and Apoptosis-Inducing Effects of Maslinic and Oleanolic Acids, Two Pentacyclic Triterpenes from Olives, on HT-29 Colon Cancer Cells. British Journal of Nutrition 2008, 100 (1), 36-43. doi.org/10.1017/S0007114508882979.
(39) Bae, G.-H.; Lee, S.-M.; Lee, E.-S.; Lee, J.-S.; Gang, J.-S. Isolation and Quantitative Analysis of Betulinic Acid and Alphitolic Acid from Zyziphi fructus. Yakhak Hoeji 1996, 40 (5), 558-562.
(40) Jannus, F.; Sainz, J.; Reyes-Zurita, F. J. Principal Bioactive Properties of Oleanolic Acid, Its Derivatives, and Analogues. Molecules 2024, 29 (14). doi.org/10.3390/molecules29143291.
(41) Yan, R.; Liu, L.; Huang, X.; Quan, Z.-S.; Shen, Q.-K.; Guo, H.-Y. Bioactivities and Structure-Activity Relationships of Maslinic Acid Derivatives: A Review. Chem Biodivers 2024, 21 (2), e202301327. doi.org/10.1002/cbdv.202301327.
(42) Lou, H.; Li, H.; Zhang, S.; Lu, H.; Chen, Q. A Review on Preparation of Betulinic Acid and Its Biological Activities. Molecules 2021, 26 (18). doi.org/10.3390/molecules26185583.
(43) Bai, L.-Y.; Chiu, C.-F.; Chiu, S.-J.; Chen, Y.-W.; Hu, J.-L.; Wu, C.-Y.; Weng, J.-R. Alphitolic Acid, an Anti-Inflammatory Triterpene, Induces Apoptosis and Autophagy in Oral Squamous Cell Carcinoma Cells, in Part, through a P53-Dependent Pathway. J Funct Foods 2015, 18, 368-378. doi.org/10.1016/j.jff.2015.07.017.
(44) Adnyana, I. K.; Tezuka, Y.; Banskota, A. H.; Tran, K. Q.; Kadota, S. Three New Triterpenes from the Seeds of Combretum quadrangulare and Their Hepatoprotective Activity. J Nat Prod 2001, 64 (3), 360-363. doi.org/10.1021/np000486x.
(45) Carocci, A.; Estrada, O. Vasorelaxant Effect of 6β-Hydroxybetulinic Acid. Revista Brasileira de Farmacognosia 2024, 34 (3), 647-653. doi.org/10.1007/s43450-024-00515-w.
(46) Estrada, O.; Contreras, W.; Acha, G.; Lucena, E.; Venturini, W.; Cardozo, A.; Alvarado-Castillo, C. Chemical Constituents from Licania cruegeriana and Their Cardiovascular and Antiplatelet Effects. Molecules 2014, 19 (12), 21215-21225. doi.org/10.3390/molecules191221215.
(47) Han, Y.; Yuan, C.; Zhou, X.; Han, Y.; He, Y.; Ouyang, J.; Zhou, W.; Wang, Z.; Wang, H.; Li, G. Anti-Inflammatory Activity of Three Triterpene from Hippophae rhamnoides L. in Lipopolysaccharide-Stimulated RAW264.7 Cells. Int J Mol Sci 2021, 22 (21). doi.org/10.3390/ijms222112009.
(48) Oliveira-Costa, J. F.; Meira, C. S.; Neves, M. V. G. das; Dos Reis, B. P. Z. C.; Soares, M. B. P. Anti-Inflammatory Activities of Betulinic Acid: A Review. Front Pharmacol 2022, 13.
(49) Chen, C.; Song, K.; Zhang, Y.; Chu, C.; Fan, B.; Song, Y.; Huang, H.; Chen, G. Biotransformation of Betulinic Acid by Circinella muscae and Cunninghamella echinulata to Discover Anti-Inflammatory Derivatives. Phytochemistry 2021, 182, 112608. doi.org/10.1016/j.phytochem.2020.112608.
(50) Blanco-Cabra, N.; Vega-Granados, K.; Moya-Andérico, L.; Vukomanovic, M.; Parra, A.; Álvarez de Cienfuegos, L.; Torrents, E. Novel Oleanolic and Maslinic Acid Derivatives as a Promising Treatment against Bacterial Biofilm in Nosocomial Infections: An in Vitro and in Vivo Study. ACS Infect Dis 2019, 5 (9), 1581-1589. doi.org/10.1021/acsinfecdis.9b00125.

1. A method for producing pentacyclic triterpenoids, the method comprising:
eliciting cultured hairy roots of Bixa orellana in a culture medium with an elicitor comprising methyl-β-cyclodextrin, methyl jasmonate, hydrogen peroxide, and magnesium chloride; and
extracting pentacyclic triterpenoids from the elicited culture medium and, optionally, the elicited hairy roots.

2. The method of claim 1, wherein the elicitor composition comprises:
between 4.5-27 g/L methyl-β-cyclodextrin;
between 1-150 μM methyl jasmonate;
between 1.5-120 mM hydrogen peroxide; and
between 0.5-10 mM magnesium chloride.

3. The method of claim 1, wherein 2α,6β-dihydroxybetulinic acid, maslinic acid, oleanolic acid, and 6β-hydroxybetulinic acid are extracted from the elicited culture medium.

4. The method of claim 3, wherein arjunolic acid and, optionally, betulinic acid, alphitolic acid, or a combination thereof, is extracted from the elicited culture medium.

5. The method of claim 1, wherein eliciting the hairy roots comprises eliciting for between 24 hours and 240 hours.

6. The method of claim 1 further comprising re-eliciting the hairy roots with the elicitor composition after extracting pentacyclic triterpenoids.

7. The method of claim 1 further comprising purifying the extracted triterpenoids.

8. The method of claim 1 further comprising derivitizing one of more extracted pentacyclic triterpenoids.

9. The method of claim 8, wherein the one or more pentacyclic triterpenoids are acetylated.

10. A composition comprising elicited, cultured hairy roots of Bixa orellana, a culture medium, an elicitor, and pentacyclic triterpenoids,
wherein the elicitor comprises methyl-β-cyclodextrin, methyl jasmonate, hydrogen peroxide, and magnesium chloride and
wherein the pentacyclic triterpenoids comprises 2α,6β-dihydroxybetulinic acid, maslinic acid, oleanolic acid, and 6β-hydroxybetulinic acid.

11. The composition of claim 10, wherein the pentacyclic triterpenoids further comprises arjunolic acid and, optionally, betulinic acid, alphitolic acid, or a combination thereof.

12. A triterpenoid-rich extract of the composition of claim 10, wherein the extract comprises 2α,6β-dihydroxybetulinic acid, maslinic acid, oleanolic acid, 6β-hydroxybetulinic acid, and an extractant.

13. The composition of claim 12, wherein the extractant comprises ethyl acetate.

14. The composition of claim 12 further comprising a pharmaceutically acceptable carrier, excipient, or diluent.

15. A method of inhibiting induction of an inflammatory marker, the method comprising contacting an immune cell with the composition according to claim 12, a pentacyclic triterpenoid obtained the composition, or a derivative of a pentacyclic triterpenoid obtained the composition.

16. The method of claim 15, wherein the immune cell is a macrophage.

17. A method of inhibiting growth or proliferation of bacteria, the method comprising contacting the bacteria with the composition according to claim 12, a pentacyclic triterpenoid obtained the composition, or a derivative of a pentacyclic triterpenoid obtained the composition.

18. The method of claim 17, wherein the bacteria is B. subtilis, E. coli, E. faecalis, E. faecium, K. pneumoniae, S. aureaus, or S. mutans.

19. The method of claim 18, wherein the bacteria is vancomycin-resistant E. faecium, vancomycin-resistant E. faecalis, or methicillin-resistant S. aureus.

20. A method of inhibiting growth or proliferation of cancer cells, the method comprising contacting the cancer cells with the composition according to claim 12, a pentacyclic triterpenoid obtained the composition, or a derivative of a pentacyclic triterpenoid obtained the composition.
